SEQUENCE LISTING (42 sequences)

SEQ ID NO: 1 - protein, 223 aa, Coronavirus SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 222 aa, Coronavirus SARS-CoV-1
RVVPTGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 3 - protein, 119 aa, Artificial Sequence
  O4C12 heavy chain variable domain
EVQLLESGGGLVQPGGSLRLSCAASGFRISDEDMGWVRQAPGKGLEWVSYIDNAGASTYYADSVKGRFTISRDNSKNTLYLQMNSLRAEDTAVYYCAKQINQGEGFDYWGQGTLVTVSS

SEQ ID NO: 4 - protein, 107 aa, Artificial Sequence
  O4C12 light chain variable domain
DIQMTQSPSSLSASVGDRVTITCRASQSISSYLNWYQQKPGKAPKLLIYYASNLQSGVPSRFSGSGSGTDFTLTISSLQPEDFATYYCQQGLGMPITFGQGTKVEIK

SEQ ID NO: 5 - protein, 121 aa, Artificial Sequence
  O4G1 heavy chain variable domain
EVQLLESGGGLVQPGGSLRLSCAASGFRFDYEDMGWVRQAPGKGLEWVSSIDSHGASTYYADSVKGRFTISRDNSKNTLYLQMNSLRAEDTAVYYCAKYSHSVSYGPFDYWGQGTLVTVSS

SEQ ID NO: 6 - protein, 107 aa, Artificial Sequence
  O4G1 light chain variable domain
DIQMTQSPSSLSASVGDRVTITCRASQYIYDDLNWYQQKPGKAPKLLIYSASSLQSGVPSRFSGSGSGTDFTLTISSLQPEDFATYYCQQTNTYPQTFGQGTKVEIK

SEQ ID NO: 7 - protein, 119 aa, Artificial Sequence; identical to SEQ ID NO: 3
  C12K-A10 heavy chain variable domain

SEQ ID NO: 8 - protein, 107 aa, Artificial Sequence
  C12K-A10 light chain variable domain
DIQMTQSPSSLSASVGDRVTITCRASQSIYDYLNWYQQKPGKAPKLLIYYASNLQSGVPSRFSGSGSGTDFTLTISSLQPEDFATYYCQQGLGMPMTFGQGTKVEIK

SEQ ID NO: 9 - protein, 119 aa, Artificial Sequence
  C12K-B12 heavy chain variable domain
EVQLLESGGGLVQPGGSLRLSCAASGFRISDEDMGWVRQAPGKGLEWVSYIDNAGASTYYADSVKGRFTISRDNSKNTLYLQMNSLRAEDTAVYYCAKSHYANPSFDYWGQGTLVTVSS

SEQ ID NO: 10 - protein, 107 aa, Artificial Sequence
  C12K-B12 light chain variable domain
DIQMTQSPSSLSASVGDRVTITCRASQDIADYLNWYQQKPGKAPKLLIYYASNLQSGVPSRFSGSGSGTDFTLTISSLQPEDFATYYCQQGLGMPITFGQGTKVEIK

SEQ ID NO: 11 - protein, 119 aa, Artificial Sequence
  C12K-D12 heavy chain variable domain
EVQLLESGGGLVQPGGSLRLSCAASGFRISDEDMGWVRQAPGKGLEWVSYIDNAGASTYYADSVKGRFTISRDNSKNTLYLQMNSLRAEDTAVYYCAKSLGPNPEFDYWGQGTLVTVSS

SEQ ID NO: 12 - protein, 107 aa, Artificial Sequence; identical to SEQ ID NO: 4
  C12K-D12 light chain variable domain

SEQ ID NO: 13 - protein, 119 aa, Artificial Sequence
  C12K-G10 heavy chain variable domain
EVQLLESGGGLVQPGGSLRLSCAASGFRISDEDMGWVRQAPGKGLEWVSYIDNAGASTYYADSVKGRFTISRDNSKNTLYLQMNSLRAEDTAVYYCAKSLNSLGVFDYWGQGTLVTVSS

SEQ ID NO: 14 - protein, 107 aa, Artificial Sequence
  C12K-G10 light chain variable domain
DIQMTQSPSSLSASVGDRVTITCRASQSIYDYLNWYQQKPGKAPKLLIYYASALQSGVPSRFSGSGSGTDFTLTISSLQPEDFATYYCQQGLGMPITFGQGTKVEIK

SEQ ID NO: 15 - protein, 121 aa, Artificial Sequence; identical to SEQ ID NO: 5
  G1K-C2 heavy chain variable domain

SEQ ID NO: 16 - protein, 107 aa, Artificial Sequence
  G1K-C2 light chain variable domain
DIQMTQSPSSLSASVGDRVTITCRASQYIYDDLNWYQQKPGKAPKLLIYSASSLQSGVPSRFSGSGSGTDFTLTISSLQPEDFATYYCQQAWPYPLTFGQGTKVEIK

SEQ ID NO: 17 - protein, 121 aa, Artificial Sequence; identical to SEQ ID NO: 5
  G1K-C4 heavy chain variable domain

SEQ ID NO: 18 - protein, 107 aa, Artificial Sequence
  G1K-C4 light chain variable domain
DIQMTQSPSSLSASVGDRVTITCRASQYISYDLNWYQQKPGKAPKLLIYSASSLQSGVPSRFSGSGSGTDFTLTISSLQPEDFATYYCQQTNTYPQTFGQGTKVEIK

SEQ ID NO: 19 - protein, 5 aa, Artificial Sequence
  O4C12 VH CDR1
DEDMG

SEQ ID NO: 20 - protein, 17 aa, Artificial Sequence
  O4C12 VH CDR2
YIDNAGASTYYADSVKG

SEQ ID NO: 21 - protein, 10 aa, Artificial Sequence
  O4C12 VH CDR3
QINQGEGFDY

SEQ ID NO: 22 - protein, 11 aa, Artificial Sequence
  O4C12 VL CDR1
RASQSISSYLN

SEQ ID NO: 23 - protein, 7 aa, Artificial Sequence
  O4C12 VL CDR2
YASNLQS

SEQ ID NO: 24 - protein, 9 aa, Artificial Sequence
  O4C12 VL CDR3
QQGLGMPIT

SEQ ID NO: 25 - protein, 5 aa, Artificial Sequence
  O4G1 VH CDR1
YEDMG

SEQ ID NO: 26 - protein, 17 aa, Artificial Sequence
  O4G1 VH CDR2
SIDSHGASTYYADSVKG

SEQ ID NO: 27 - protein, 12 aa, Artificial Sequence
  O4G1 VH CDR3
YSHSVSYGPFDY

SEQ ID NO: 28 - protein, 11 aa, Artificial Sequence
  O4G1 VL CDR1
RASQYIYDDLN

SEQ ID NO: 29 - protein, 7 aa, Artificial Sequence
  O4G1 VL CDR2
SASSLQS

SEQ ID NO: 30 - protein, 9 aa, Artificial Sequence
  O4G1 VL CDR3
QQTNTYPQT

SEQ ID NO: 31 - protein, 11 aa, Artificial Sequence
  C12K-A10 VL CDR1
RASQSIYDYLN

SEQ ID NO: 32 - protein, 9 aa, Artificial Sequence
  C12K-A10 VL CDR3
QQGLGMPMT

SEQ ID NO: 33 - protein, 10 aa, Artificial Sequence
  C12K-B12 VH CDR3
SHYANPSFDY

SEQ ID NO: 34 - protein, 11 aa, Artificial Sequence
  C12K-B12 VL CDR1
RASQDIADYLN

SEQ ID NO: 35 - protein, 10 aa, Artificial Sequence
  C12K-D12 VH CDR3
SLGPNPEFDY

SEQ ID NO: 36 - protein, 10 aa, Artificial Sequence
  C12K-G10 VH CDR3
SLNSLGVFDY

SEQ ID NO: 37 - protein, 11 aa, Artificial Sequence
  C12K-G10 VL CDR1
RASQSIYDYLN

SEQ ID NO: 38 - protein, 7 aa, Artificial Sequence
  C12K-G10 VL CDR2
YASALQS

SEQ ID NO: 39 - protein, 9 aa, Artificial Sequence
  G1K-C2 VL CDR3
QQAWPYPLT

SEQ ID NO: 40 - protein, 11 aa, Artificial Sequence
  G1K-C4 VL CDR1
RASQYISYDLN

SEQ ID NO: 41 - protein, 223 aa, Artificial Sequence
  Bat-RaTG13
RVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTNLVKNKCVNF

SEQ ID NO: 42 - protein, 223 aa, Artificial Sequence
  Pangolin-Cov-RBD
RVQPTESIVRFPNITNLCPFGEVFNATTFASVYAWNRKRISNCVADYSVLYNSTSFSTFKCYGVSPTKLNDLCFTNVYADSFVVRGDEVRQIAPGQTGRIAGYNYKLPDDFTGCVIAWNSNNLDSKVGGNYNYLYRLFRKSNLKPFERDISTEIYQAGSTPCNGVEGFNCYFPLQSYGFHPTNGVGYQPYRVVVLSFELLNAPATVCGPKQSTNLVKNKCVNF

(19) United States
(12) Patent Application Publication
(10) Pub. No.: US 2024/0043506 A1
(43) Pub. Date: Feb. 8, 2024

(54) SARS-COV-2 ANTIBODIES

(71) Applicant: GARVAN INSTITUTE OF MEDICAL RESEARCH, Darlinghurst, New South Wales (AU)

(72) Inventors: Ohan MAZIGI, Petersham, New South Wales (AU); David LANGLEY, Marsfield, New South Wales (AU); Peter SCHOFIELD, Carlton, New South Wales (AU); Daniel CHRIST, Annandale, New South Wales (AU)

(21) Appl. No.: 18/258,968

(22) PCT Filed: Dec. 23, 2021
(86) PCT No.: PCT/AU2021/051550

§ 371 Date: Jun. 22, 2023

(30) Foreign Application Priority Data
Dec. 23, 2020 (AU) 2020904813

(51) Int. Cl. C07K 16/10; A61P 31/14; G01N 33/569

(52) U.S. Cl. CPC C07K 16/1003; A61P 31/14; G01N 33/56983; C07K 2317/92; C07K 2317/565; C07K 2317/622; C07K 2317/524; C07K 2317/526; G01N 2800/26

(57) ABSTRACT

The present disclosure is directed to isolated or recombinant antigen binding proteins, such as antibodies, which bind to coronavirus spike (S) protein receptor binding domain (RBD), including S protein RBD from severe acute respiratory syndrome coronavirus 2 (SARS-CoV-2). The present disclosure is also directed to the use of the isolated or recombinant proteins as therapeutic, prophylactic and/or diagnostic agents for respiratory conditions associated with coronavirus infection, such as infection by SARS-CoV-2. The present disclosure is also related to nucleic acid sequences which encode said antigen binding proteins and their expression in recombinant host cells.

### CROSS-REFERENCE TO RELATED APPLICATIONS

The present application claims priority from Australian Provisional Patent Application No 2020904813 filed on 23 Dec. 2020, the content of which is incorporated herein by reference in its entirety.

### FIELD OF THE INVENTION

The present disclosure is directed to isolated or recombinant antigen binding proteins, such as antibodies, which bind to coronavirus spike (S) protein receptor binding domain (RBD), including S protein RBD from severe acute respiratory syndrome coronavirus 2 (SARS-CoV-2). The present disclosure is also directed to the use of the isolated or recombinant proteins as therapeutic, prophylactic and/or diagnostic agents for respiratory conditions associated with coronavirus infection, such as infection by SARS-CoV-2. The present disclosure is also related to nucleic acid sequences which encode said antigen binding proteins and their expression in recombinant host cells.

### BACKGROUND OF THE INVENTION

Similar to other severe acute respiratory syndrome (SARS) viruses, such as SARS-CoV and MERS-CoV, SARS-CoV-2 is an enveloped, single-stranded, and positive (+)-sense

RNA virus, belonging to the beta-CoV genera in the family Coronaviridae. Coronavirus (CoV) disease 2019 (COVID-19) caused by SARS-CoV-2 (also known as 2019-nCoV) is threatening global public health, social stability, and economic development. Different from SARS-CoV and MERS-CoV, the SARS-CoV-2 virus is characterized by its rapid spread and virulent human-to-human transmission. SARS-CoV-2 was first reported in humans in Wuhan, China in December 2019. By March 2020, the World Health Organisation (WHO) declared the outbreak of the virus a worldwide pandemic. At the time of writing this, there has been 75 million cases of SARS-CoV-2 infection reported globally, and 1.7 M deaths associated with SARS-CoV-2; all within a period of less than 12 months.

The SARS-CoV-2 pandemic has seen an unprecedented focus on the development of vaccines against coronavirus, with more than 200 vaccines in the pipeline and over 30 vaccines in clinical trial. The majority of vaccines in development attempt to provoke an immune response against the SARS-COV-2 spike protein (or S protein). Whilst a limited number of vaccines have received emergency use authorisation in certain jurisdictions (including USA, UK, Canada, Mexico, Singapore, China, Russia and the UAE), for many jurisdictions no vaccines or treatments are as yet available. It also remains unclear whether those vaccines which have received emergency use authorisation will be effective therapeutic and/or preventative agents against SARS-CoV-2 and/or COVID-19.

There is therefore a need to develop further medical interventions, including neutralizing antibodies (nAbs), for the prevention, treatment and diagnosis of infection by SARS-CoV-2.

### SUMMARY

The present disclosure is based, inter alia, on the inventors' identification of antigen binding proteins against coronavirus (CoV) spike (S) protein receptor binding domain (RBD), including severe acute respiratory syndrome coronavirus 2 (SARS-CoV-2). Initial screens performed by the inventors identified antigen-binding proteins comprising an antibody variable region that binds SARS-CoV-2 S protein RBD. Subsequent affinity maturation of selected antigen binding proteins resulted in the identification of variant antigen binding proteins capable of binding S protein RBD of both SARS-CoV-2 and SARS-CoV-1, leading the inventors to conclude that the antigen binding proteins bind a conserved epitope with the coronavirus S protein RBD. Specifically, the inventors have identified antigen binding proteins which bind an epitope of the CoV S protein RBD comprising, inter alia, an isoleucine (I) at position 150 (i.e., 1501) numbered relative to the S protein RBD amino acid sequence set forth in SEQ ID NO: 1. The inventors have shown that the antigen binding protein identified in their screens are capable of binding CoV S protein RBD e.g., such as SARS-CoV-2 S protein RBD, with good affinity. The inventors have also found that these antigen binding proteins are capable of neutralising coronavirus infection of mammalian cells. These findings provide a basis for methods of treating, preventing and/or delaying progression of a disease or disorder caused by infection with a coronavirus (e.g., a disease caused by a SARS-CoV-2 infection, such as COVID-19 or ARDS) in a subject. These findings also provide a basis for methods of detecting the presence or absence of a CoV S protein RBD (e.g., a SARS-CoV-2 S protein RBD) in a sample, such as may be required in a diagnostic test.

Accordingly, the present disclosure provides an antigen-binding protein comprising an antibody variable region which binds to a CoV S protein RBD, wherein the antibody variable region binds to an epitope of the CoV S protein RBD comprising at least residue 150 (e.g., 1501) numbered relative to the SARS CoV-2 S protein RBD amino acid sequence set forth in SEQ ID NO: 1.

In one example, the antibody variable region binds to an isoleucine (I) at position 150 within the epitope of the CoV S protein RBD, wherein residue 1501 is numbered relative to the SARS-CoV-2 S protein RBD amino acid sequence set forth in SEQ ID NO: 1.

In one example, the antigen-binding protein binds SARS-CoV-2 S protein RBD and SARS-CoV-1 S protein RBD.

In one example, the antigen-binding protein binds SARS-CoV-2 S protein RBD with an equilibrium binding constant (K_{D}) of about 60 nM or better. For example, the antigen-binding protein binds SARS-CoV-2 S protein RBD with a KD of about 60 nM or better, for example about 50 nM, or about 40 nM, or about 30 nM, or about 20 nM, or about 10 nM, or about 1 nM or better. For example, the antigen-binding protein binds SARS-CoV-2 S protein

RBD with a KD of about 30 nM or better. For example, the antigen-binding protein binds SARS-CoV-2 S protein RBD with a KD of about 25 nM or better. For example, the antigen-binding protein binds SARS-CoV-2 S protein RBD with a KD of about 20 nM or better. For example, the antigen-binding protein binds SARS-CoV-2 S protein RBD with a KD of about 15 nM or better. For example, the antigen-binding protein binds SARS-CoV-2 S protein

RBD with a KD of about 10 nM or better. For example, the antigen-binding protein binds SARS-CoV-2 S protein RBD with a KD of about 5 nM or better. For example, the antigen-binding protein binds SARS-CoV-2 S protein RBD with a KD of about 1 nM or better.

In one example, the antigen-binding protein of the disclosure neutralises coronavirus infection of mammalian cells. In one example, the antigen-binding protein neutralises SARS-CoV-2 infection of mammalian cells. For example, the antigen-binding protein neutralises SARS-CoV-2 infection of Vero E6 cells with a half-maximal inhibitory concentration (IC50) of about 50 mg/mL or better. For example, the antigen-binding protein neutralises SARS-CoV-2 infection of Vero E6 cells with an IC50 of about 50 mg/mL, or about 40 mg/mL, or about 30 mg/mL, or about 20 mg/mL, or about 10 mg/mL, or about 1 mg/mL or better. For example, the antigen-binding protein neutralises SARS-CoV-2 infection of Vero E6 cells with an IC50 of about 25 mg/mL or better. For example, the antigen-binding protein neutralises SARS-CoV-2 infection of Vero E6 cells with an IC50 of about 20 mg/mL or better. For example, the antigen-binding protein neutralises SARS-CoV-2 infection of Vero E6 cells with an IC50 of about 15 mg/mL or better. For example, the antigen-binding protein neutralises SARS-CoV-2 infection of Vero E6 cells with an IC50 of about 10 mg/mL or better. For example, the antigen-binding protein neutralises SARS-CoV-2 infection of Vero E6 cells with an IC50 of about 5 mg/mL or better. For example, the antigen-binding protein neutralises SARS-CoV-2 infection of Vero E6 cells with an IC50 of about 1 μg/mL or better.

Methods of determining neutralising activity of an antigen-binding protein will be apparent to the skilled person and/or are described herein. Exemplary assays include a Vero microneutralisation assay, a surrogate viral neutralisation test (sVNT) and a psuedovirus neutralisation assay (PsV; using e.g., 293T or HeLa-ACE2 cell lines).

In one example, the antigen-binding protein of the disclosure comprises an antibody variable region which binds to the same epitope within a coronavirus S protein RBD as that bound by an antibody selected from:
(i) an antibody comprising a heavy chain variable domain (V_{H}) comprising the sequence set forth in SEQ ID NO: 3 and a light chain variable domain (V_{L}) comprising the sequence set forth in SEQ ID NO: 4 (O4C12);
(ii) an antibody comprising a V_{H} comprising the sequence set forth in SEQ ID NO: 7 and a V_{L} comprising the sequence set forth in SEQ ID NO: 8 (C12K-A10);
(iii) an antibody comprising a V_{H} comprising the sequence set forth in SEQ ID NO: 9 and a V_{L} comprising the sequence set forth in SEQ ID NO: 10 (C12K-B12);
(iv) an antibody comprising a V_{H} comprising the sequence set forth in SEQ ID NO: 11 and a V_{L} comprising the sequence set forth in SEQ ID NO: 12 (C12K-D12);
(v) an antibody comprising a V_{H} comprising the sequence set forth in SEQ ID NO: 13 and a V_{L} comprising the sequence set forth in SEQ ID NO: 14 (C12K-G10);
(vi) an antibody comprising a V_{H} comprising the sequence set forth in SEQ ID NO: 5 and a V_{L} comprising the sequence set forth in SEQ ID NO: 6 (O4G1);
(vii) an antibody comprising a V_{H} comprising the sequence set forth in SEQ ID NO: 15 and a V_{L} comprising the sequence set forth in SEQ ID NO: 16 (G1K-C2); and
(viii) an antibody comprising a V_{H} comprising the sequence set forth in SEQ ID NO: 17 and a V_{L} comprising the sequence set forth in SEQ ID NO: 18 (G1K-C4).

In one example, the antigen-binding protein comprises an antibody variable region which competitively inhibits binding of an antibody comprising a V_{H} comprising a sequence set forth in SEQ ID NO: 3 and a V_{L} comprising a sequence set forth in SEQ ID NO: 4 to the SARS-CoV-2 S protein RBD.

In one example, the antigen-binding protein comprises an antibody variable region which competitively inhibits binding of an antibody comprising a V_{H} comprising a sequence set forth in SEQ ID NO: 7 and a V_{L} comprising a sequence set forth in SEQ ID NO: 8 to the SARS-CoV-2 S protein RBD.

In one example, the antigen-binding protein comprises an antibody variable region which competitively inhibits binding of an antibody comprising a V_{H} comprising a sequence set forth in SEQ ID NO: 9 and a V_{L} comprising a sequence set forth in SEQ ID NO: 10 to the SARS-CoV-2 S protein RBD.

In one example, the antigen-binding protein comprises an antibody variable region which competitively inhibits binding of an antibody comprising a V_{H} comprising a sequence set forth in SEQ ID NO: 11 and a V_{L} comprising a sequence set forth in SEQ ID NO: 12 to the SARS-CoV-2 S protein RBD.

In one example, the antigen-binding protein comprises an antibody variable region which competitively inhibits binding of an antibody comprising a V_{H} comprising a sequence set forth in SEQ ID NO: 13 and a V_{L} comprising a sequence set forth in SEQ ID NO: 14 to the SARS-CoV-2 S protein RBD.

In one example, the antigen-binding protein comprises an antibody variable region which competitively inhibits binding of an antibody comprising a V_{H} comprising a sequence set forth in SEQ ID NO: 5 and a V_{L} comprising a sequence set forth in SEQ ID NO: 6 to the SARS-CoV-2 S protein RBD.

In one example, the antigen-binding protein comprises an antibody variable region which competitively inhibits binding of an antibody comprising a V_{H} comprising a sequence set forth in SEQ ID NO: 15 and a V_{L} comprising a sequence set forth in SEQ ID NO: 16 to the SARS-CoV-2 S protein RBD.

In one example, the antigen-binding protein comprises an antibody variable region which competitively inhibits binding of an antibody comprising a V_{H} comprising a sequence set forth in SEQ ID NO: 17 and a V_{L} comprising a sequence set forth in SEQ ID NO: 18 to the SARS-CoV-2 S protein RBD.

Methods of determining competitive inhibition will be apparent to the skilled person and/or are described herein.

In one example, the antigen-binding protein of the disclosure comprises an antibody variable region comprising:
(i) a V_{H} comprising complementarily determining region (CDR) 1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 22, 23 and 24 respectively (O4C12);
(ii) a V_{H} comprising CDR1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 31, 23 and 32 respectively (C12K-A10);
(iii) a V_{H} comprising CDR1, CDR2 and CDR3 comprising the sequences set forth in SEQ ID NOs: 19, 20 and 33 respectively, and a V_{L} comprising CDR1, CDR2 and CDR3 comprising the sequences set forth in SEQ ID NOs: 34, 23 and 24 respectively (C12K-B12);
(iv) a V_{H} comprising CDR1, CDR2 and CDR3 comprising the sequences set forth in SEQ ID NOs: 19, 20 and 35 respectively, and a V_{L} comprising CDR1, CDR2 and CDR3 comprising the sequences set forth in SEQ ID NOs: 22, 23 and 24 respectively C12K-D12);
(v) a V_{H} comprising CDR1, CDR2 and CDR3 comprising the sequences set forth in SEQ ID NOs: 19, 20 and 36 respectively, and a V_{L} comprising CDR1, CDR2 and CDR3 comprising the sequences set forth in SEQ ID NOs: 37, 38 and 24 respectively (C12K-G10);
(vi) a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 30 respectively (O4G1);
(vii) a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 39 respectively (G1K-C2); or
(viii) a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 40, 29 and 30 respectively (G1K-C4).

In one example, the antigen-binding protein comprises a V_{H} comprising CDR1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 22, 23 and 24 respectively (O4C12).

In one example, the antigen-binding protein comprises a V_{H} comprising CDR1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 31, 23 and 32 respectively (C12K-A10).

In one example, the antigen-binding protein comprises a V_{H} comprising CDR1, CDR2 and CDR3 comprising the sequences set forth in SEQ ID NOs: 19, 20 and 33 respectively, and a V_{L} comprising CDR1, CDR2 and CDR3 comprising the sequences set forth in SEQ ID NOs: 34, 23 and 24 respectively (C12K-B12).

In one example, the antigen-binding protein comprises a V_{H} comprising CDR1, CDR2 and CDR3 comprising the sequences set forth in SEQ ID NOs: 19, 20 and 35 respectively, and a V_{L} comprising CDR1, CDR2 and CDR3 comprising the sequences set forth in SEQ ID NOs: 22, 23 and 24 respectively C12K-D12).

In one example, the antigen-binding protein comprises a V_{H} comprising CDR1, CDR2 and CDR3 comprising the sequences set forth in SEQ ID NOs: 19, 20 and 36 respectively, and a V_{L} comprising CDR1, CDR2 and CDR3 comprising the sequences set forth in SEQ ID NOs: 37, 38 and 24 respectively (C12K-G10).

In one example, the antigen-binding protein comprises a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 30 respectively (O4G1).

In one example, the antigen-binding protein comprises a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 39 respectively (G1K-C2).

In one example, the antigen-binding protein comprises a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 40, 29 and 30 respectively (G1K-C4).

In one example, the antigen-binding protein of the disclosure comprises an antibody variable region comprising:
(i) a V_{H} comprising a sequence which is at least 90% identical to the sequence set forth in SEQ ID NO: 3 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical to the sequence set forth in SEQ ID NO: 4 provided that the V_{L} comprises a CDR1 set forth in SEQ ID NO: 22, a CDR2 set forth in SEQ ID NO: 23 and a CDR3 set forth in SEQ ID NO: 24 (O4C12);
(ii) a V_{H} comprising a sequence which is at least 90% identical to the sequence set forth in SEQ ID NO: 7 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical to the sequence set forth in SEQ ID NO: 8 provided that the V_{L} comprises a CDR1 set forth in SEQ ID NO: 31, a CDR2 set forth in SEQ ID NO: 23 and a CDR3 set forth in SEQ ID NO: 32 (C12K-A10);
(iii) a V_{H} comprising a sequence which is at least 90% identical to the sequence set forth in SEQ ID NO: 9 provided that the V_{H} comprises a CDR1 set forth in SEQ ID NO: 19, a CDR2 set forth in SEQ ID NO: 20 and a CDR3 set forth in SEQ ID NO: 33, and a V_{L} comprising a sequence which is at least 90% identical to the sequence set forth in SEQ ID NO: 10 provided that the V_{L} comprises a CDR1 set forth in SEQ ID NO: 33, a CDR2 set forth in SEQ ID NO: 23 and a CDR3 set forth in SEQ ID NO: 24 (C12K-B12);
(iv) a V_{H} comprising a sequence which is at least 90% identical to the sequence set forth in SEQ ID NO: 11 provided that the V_{H} comprises a CDR1 set forth in SEQ ID NO: 19, a CDR2 set forth in SEQ ID NO: 20 and a CDR3 set forth in SEQ ID NO: 35, and a V_{L} comprising a sequence which is at least 90% identical to the sequence set forth in SEQ ID NO: 12 provided that the V_{L} comprises a CDR1 set forth in SEQ ID NO: 22, a CDR2 set forth in SEQ ID NO: 23 and a CDR3 set forth in SEQ ID NO: 24 (C12K-D12);
(v) a V_{H} comprising a sequence which is at least 90% identical to the sequence set forth in SEQ ID NO: 13 provided that the V_{H} comprises a CDR1 set forth in SEQ ID NO: 19, a CDR2 set forth in SEQ ID NO: 20 and a CDR3 set forth in SEQ ID NO: 36, and a V_{L} comprising a sequence which is at least 90% identical to the sequence set forth in SEQ ID NO: 14 provided that the V_{L} comprises a CDR1 set forth in SEQ ID NO: 37, a CDR2 set forth in SEQ ID NO: 38 and a CDR3 set forth in SEQ ID NO: 24 (C12K-G10);
(vi) a V_{H} comprising a sequence which is at least 90% identical to the sequence set forth in SEQ ID NO: 5 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical to the sequence set forth in SEQ ID NO: 6 provided that the V_{L} comprises a CDR1 set forth in SEQ ID NO: 28, a CDR2 set forth in SEQ ID NO: 29 and a CDR3 set forth in SEQ ID NO: 30 (O4G1);
(vii) a V_{H} comprising a sequence which is at least 90% identical to the sequence set forth in SEQ ID NO: 15 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical to the sequence set forth in SEQ ID NO: 16 provided that the V_{L} comprises a CDR1 set forth in SEQ ID NO: 28, a CDR2 set forth in SEQ ID NO: 29 and a CDR3 set forth in SEQ ID NO: 39 (G1K-C2); or
(viii) a V_{H} comprising a sequence which is at least 90% identical to the sequence set forth in SEQ ID NO: 17 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical to the sequence set forth in SEQ ID NO: 18 provided that the V_{L} comprises a CDR1 set forth in SEQ ID NO: 40, a CDR2 set forth in SEQ ID NO: 29 and a CDR3 set forth in SEQ ID NO: 30 (G1K-C4). In one example, the antigen binding protein comprises a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 3 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 4 provided that the V_{L} comprises a CDR1 set forth in SEQ ID NO: 22, a CDR2 set forth in SEQ ID NO: 23 and a CDR3 set forth in SEQ ID NO: 24 (O4C12). For example, the antigen binding protein may comprise a V_{H} comprising the sequence set forth in SEQ ID NO: 3 and a V_{L} comprising the sequence set forth in SEQ ID NO: 4 (O4C12).

In one example, the antigen binding protein comprises a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 7 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 8 provided that the V_{L} comprises a CDR1 set forth in SEQ ID NO: 31, a CDR2 set forth in SEQ ID NO: 23 and a CDR3 set forth in SEQ ID NO: 32 (C12K-A10). For example, the antigen binding protein may comprise a V_{H} comprising the sequence set forth in SEQ ID NO: 7 and a V_{L} comprising the sequence set forth in SEQ ID NO: 8 (C12K-A10).

In one example, the antigen binding protein comprises a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 9 provided that the V_{H} comprises a CDR1 set forth in SEQ ID NO: 19, a CDR2 set forth in SEQ ID NO: 20 and a CDR3 set forth in SEQ ID NO: 33, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 10 provided that the V_{L} comprises a CDR1 set forth in SEQ ID NO: 33, a CDR2 set forth in SEQ ID NO: 23 and a CDR3 set forth in SEQ ID NO: 24 (C12K-B12). For example, the antigen binding protein may comprise a V_{H} comprising the sequence set forth in SEQ ID NO: 9 and a V_{L} comprising the sequence set forth in SEQ ID NO: 10 (C12K-B12).

In one example, the antigen binding protein comprises a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 11 provided that the V_{H} comprises a CDR1 set forth in SEQ ID NO: 19, a CDR2 set forth in SEQ ID NO: 20 and a CDR3 set forth in SEQ ID NO: 35, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 12 provided that the V_{L} comprises a CDR1 set forth in SEQ ID NO: 22, a CDR2 set forth in SEQ ID NO: 23 and a CDR3 set forth in SEQ ID NO: 24 (C12K-D12). For example, the antigen binding protein may comprise a V_{H} comprising the sequence set forth in SEQ ID NO: 11 and a V_{L} comprising the sequence set forth in SEQ ID NO: 12 (C12K-D12).

In one example, the antigen binding protein comprises a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 13 provided that the V_{H} comprises a CDR1 set forth in SEQ ID NO: 19, a CDR2 set forth in SEQ ID NO: 20 and a CDR3 set forth in SEQ ID NO: 36, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 14 provided that the V_{L} comprises a CDR1 set forth in SEQ ID NO: 37, a CDR2 set forth in SEQ ID NO: 38 and a CDR3 set forth in SEQ ID NO: 24 (C12K-G10). For example, the antigen binding protein may comprise a V_{H} comprising the sequence set forth in SEQ ID NO: 13and a V_{L} comprising the sequence set forth in SEQ ID NO: 14 (C12K-G10).

In one example, the antigen binding protein comprises a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 5 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 6 provided that the V_{L} comprises a CDR1 set forth in SEQ ID NO: 28, a CDR2 set forth in SEQ ID NO: 29 and a CDR3 set forth in SEQ ID NO: 30 (O4G1). For example, the antigen binding protein may comprise a V_{H} comprising the sequence set forth in SEQ ID NO: 5 and a V_{L} comprising the sequence set forth in SEQ ID NO: 6 (O4G1).

In one example, the antigen binding protein comprises a Vx comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 15 provided that the Vx comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 16 provided that the V_{L} comprises a CDR1 set forth in SEQ ID NO: 28, a CDR2 set forth in SEQ ID NO: 29 and a CDR3 set forth in SEQ ID NO: 39 (G1K-C2). For example, the antigen binding protein may comprise a V_{H} comprising the sequence set forth in SEQ ID NO: 15 and a V_{L} comprising the sequence set forth in SEQ ID NO: 16 (G1K-C2).

In one example, the antigen binding protein comprises a Vx comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 17 provided that the Vx comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 18 provided that the V_{L} comprises a CDR1 set forth in SEQ ID NO: 40, a CDR2 set forth in SEQ ID NO: 29 and a CDR3 set forth in SEQ ID NO: 30 (G1K-C4). For example, the antigen binding protein may comprise a V_{H} comprising the sequence set forth in SEQ ID NO: 17 and a V_{L} comprising the sequence set forth in SEQ ID NO: 18 (G1K-C4). In one example, the antigen-binding protein comprises a fragment variable (Fv).

In one example, the antigen-binding protein is selected from the group consisting of:
(i) a single chain fragment variable (Fv) fragment (scFv);
(ii) a dimeric scFv (di-scFv);
(iii) a diabody;
(iv) a triabody;
(v) a tetrabody;
(vi) a fragment antigen binding (Fab);
(vii) a F(ab′)₂;
(viii) a Fv;
(ix) one of (i) to (viii) linked to a constant region of an antibody, a constant fragment (Fc) or a heavy chain constant domain (CH)₂ and/or C_{H}3; or
(x) an antibody. For example, the antigen-binding protein comprises a V_{H} and a V_{L} wherein the V_{H} and the V_{L} are in a single polypeptide chain and the protein is selected from the group consisting of:
(a) a scFv;
(b) a di-scFv; and
(c) one of (a) or (b) linked to a constant region of an antibody, a Fc or a C_{H}2 and/or

C_{H}3.

In another example, the antigen-binding protein comprises a V_{H} and a V_{L}, wherein the V_{H} and the V_{L} are in separate polypeptide chains and the protein is selected from the group consisting of:
(a) a diabody;
(b) a triabody;
(c) a tetrabody;
(d) a Fab;
(e) a F(ab′)₂;
(f) a Fv;
(g) one of (a) to (f) linked to a constant region of an antibody, a Fc or a C_{H}2 and/or C_{H}3; and
(h) an antibody.

In one example, the antigen-binding protein is a an antibody. Exemplary antibodies are full length and/or naked antibodies. For example, the protein is an anti-SARS-CoV-2 antibody. In one example, the anti-SARS-CoV-2 antibody is a monoclonal anti-SARS-CoV-2 antibody.

In one example, the antigen-binding protein is chimeric, CDR grafted, de-immunized, humanized, synhumanized, primatized or human.

In one example, the antigen-binding protein is a human antibody.

In one example, the antigen-binding protein is conjugated to a compound. For example, the antigen-binding protein may be conjugated to a compound selected from the group consisting of a detectable label, a therapeutic compound, a nucleic acid, a peptide, a protein, a compound that increases the half-life of the protein in a subject and mixtures thereof. In one particular example, the antigen-binding protein is conjugated to a detectable label. An exemplary detectable label is selected from the group consisting of: a radiolabel, a fluorescent label, an enzymatic label and an imaging agent.

The present disclosure also provides one or more nucleic acids encoding the antigen-binding protein described herein.

The present disclosure further provides an expression construct comprising the one or more nucleic acids of the disclosure operably linked to a promoter. Such an expression construct can be in a vector, e.g., a plasmid.

In accordance with an example in which the expression construct encodes a single polypeptide that forms an antigen-binding protein of the disclosure, the expression construct may comprise a promoter linked to a nucleic acid encoding that polypeptide chain.

In accordance with examples in which the expression construct encodes multiple polypeptides that collectively form an antigen-binding protein of the disclosure, an expression construct of the disclosure may comprise a nucleic acid encoding one of the polypeptides (e.g., comprising a V_{H}) operably linked to a promoter and a nucleic acid encoding another of the polypeptides (e.g., comprising a V_{L}) operably linked to another promoter.

In accordance with another example wherein multiple polypeptides collectively form an antigen-binding protein of the disclosure, the multiple polypeptides may be expressed by separate expression constructs. Accordingly, the present disclosure contemplate a plurality of expression constructs, wherein one encodes a first polypeptide (e.g., comprising a V_{H}) and another encodes a second polypeptide (e.g., comprising a V_{L}). For example, the present disclosure may provide a plurality of expression constructs comprising:
(i) a first expression construct comprising a nucleic acid encoding a polypeptide (e.g., comprising a V_{H} operably linked to a promoter); and
(ii) a second expression construct comprising a nucleic acid encoding a polypeptide (e.g., comprising a V_{L} operably linked to a promoter),
   wherein the first and second polypeptides associate to form a protein comprising an antibody variable region. The expression constructs may be provided separately or together.

The present disclosure also provides a host cell comprising the one or more nucleic acids of the disclosure and which is capable of expressing an antigen-binding protein of the disclosure. Exemplary host cells are isolated cells. For example, the disclosure provides use of an isolated cell for preparing the antigen-binding protein of the disclosure

The present disclosure also provides a pharmaceutical composition comprising the protein and an acceptable carrier. In one example, the carrier is pharmaceutically acceptable.

The present disclosure also provides the antigen-binding protein, the nucleic acid(s), expression construct(s) or the composition of the disclosure for use in treating or preventing coronavirus infection in a subject. In one example, the present disclosure provides the antigen-binding protein, the nucleic acid(s), expression construct(s) or the composition of the disclosure for use in treating coronavirus infection in a subject. In one example, the present disclosure provides the antigen-binding protein, the nucleic acid(s), expression construct(s) or the composition of the disclosure for use in preventing coronavirus infection in a subject.

The present disclosure also provides a method for treating or preventing infection with a CoV in a subject in need thereof, the method comprising administering to the subject the antigen-binding protein, the nucleic acid(s), expression construct(s) or the composition of the disclosure. In one example, the present disclosure provides a method for treating infection with a CoV in a subject. In another example, the present disclosure provides a method for preventing infection with a CoV in a subject.

The present disclosure also provides use of the antigen-binding protein, the nucleic acid(s), expression construct(s), host cell or composition of the disclosure in the manufacture of a medicament for treating or preventing CoV infection in a subject in need thereof. For example, the disclosure provides use of the antigen-binding protein, the nucleic acid(s), expression construct(s), host cell or composition of the disclosure in the manufacture of a medicament for treating CoV infection in a subject in need thereof. For example, the disclosure provides use of the antigen-binding protein, the nucleic acid(s), expression construct(s), host cell or composition of the disclosure in the manufacture of a medicament for preventing CoV infection in a subject in need thereof.

The present disclosure also provides for use of an antigen-binding protein, a nucleic acid or expression construct or a composition to treat or prevent infection with a CoV. in a subject.

In one example, the coronavirus is SARS-CoV-2. In another example, the coronavirus is SARS-CoV-1. In another example, the coronavirus is MERS-CoV.

In one example, the subject is suffering from a respiratory infection with coronavirus (i.e., the subject is in need of treatment). For example, the respiratory infection is an infection with SARS-CoV-2 (i.e., coronavirus disease 2019 (COVID-19)). For example, treatment of the subject may occur following detection of infection with a diagnostic test.

In one example, treatment with the antigen-binding protein of the disclosure neutralises the CoV infection in the subject.

In another example, the subject is at risk of being infected with SARS-CoV-2 and developing COVID-19. A subject at risk may be one or more of the following: over 70 years of age, immunosuppressed, immunedeficient, receiving immunosuppressive therapy, received a bone-marrow transplant in past 12 months, suffering from a blood cancer, receiving treatment for cancer, suffer from chronic kidney failure, suffer from heart disease, suffer from chronic lung disease, suffer from diabetes, suffer from chronic liver disease, and any combination thereof,

In one example, treatment or prevention comprises administering the antigen-binding protein, the nucleic acid(s), expression construct(s), or composition of the disclosure in an amount sufficient to reduce the severity of, or prevent onset of, one or more symptoms of a SARS-CoV-2 infection or COVID-19. Symptoms of a SARS-CoV-2 infection or COVID-19 will be apparent to the skilled person and/or are described herein.

In one example, treatment or prevention comprises administering a single dose of the antigen-binding protein, the nucleic acid(s), expression construct(s), or composition of the disclosure to the subject.

In another example, treatment or prevention comprises administering a multiple doses of the antigen-binding protein, the nucleic acid(s), expression construct(s), or composition of the disclosure to the subject at different time points. For example a first and a second and/or subsequent dose may be administered at defined intervals, for example about 4-6 weeks apart, or about 6-12 weeks apart, or about 12-18 weeks apart or about 18-24 weeks apart.

In accordance with any method described herein, the subject is a mammal, for example a primate, such as a human.

The present disclosure also provides a method of detecting the presence or absence of a CoV S protein RBD in a sample, said method comprising:
(i) contacting the sample with an antigen-binding protein of the disclosure; and
(ii) analysing the sample for binding between CoV S protein RBD and the antigen-binding protein. Binding of the antigen-binding protein to the CoV S protein RBD indicates the present of CoV in the sample.

The present disclosure also provides a method of diagnosing CoV infection in a subject, the method comprising:
(i) performing a method of detecting the presence or absence of a CoV S protein RBD in a sample as described herein on a sample obtained from a subject to determine the presence or absence of CoV S protein RBD in the sample; and
(ii) diagnosing whether or not the subject is infected with coronavirus based on the presence or absence of the CoV S protein RBD in the sample. Detection of binding of the antigen-binding protein to the CoV S protein RBD indicates that the subject is positive for CoV infection.

In one example, the method of detection or diagnosis is performed in vitro and the sample is, or has been obtained from, a nasopharyngeal swab, a oropharyngeal swab, a nasal aspirate, a nasal wash, saliva, sputum, tracheal aspirate or bronchoalveolar lavage (BAL).

In one example, the CoV which is detected in the sample is SARS CoV-2.

In one example, the CoV which is detected in the sample is SARS CoV-1.

In one example, the CoV which is detected in the sample is MERS-CoV.

The present disclosure also provides for use of an antigen-binding protein to detect the presence of absence of CoV S protein in a sample. In some examples, the antigen-binding protein is detectably labelled. Suitable detectable labels are known to the skilled person and described herein.

The present disclosure also provides a kit comprising an antigen-binding protein, a nucleic acid, expression construct or composition of the disclosure packaged with instructions for use in treating or preventing infection with CoV (e.g., a SARS-CoV-2 infection) in a subject according to the method described herein.

In one example, the kit further comprises a delivery system. For example, the antigen-binding protein, a nucleic acid, expression construct or composition of the disclosure may be supplied in a vial. For example, the antigen-binding protein, a nucleic acid, expression construct or composition of the disclosure may be supplied in a syringe.

In another example, the kit may comprise a separate pharmaceutically acceptable carrier or diluent.

The present disclosure also provides a kit comprising an antigen-binding protein of the disclosure packaged with instructions for use in detecting the presence or absence of a CoV S protein RBD in a sample according to the method described herein.

In one example, the antigen-binding protein of the disclosure is detectably labelled. Suitable detectable labels are known to the skilled person and described herein.

In one example, the kit comprises a positive control for CoV and/or a negative control.

### BRIEF DESCRIPTION OF THE ACCOMPANYING DRAWINGS

FIG. 1 shows phage display selection for SARS-CoV-2 antibodies over 4 rounds using biotinylated SARS-CoV-2 RBD and the Garvan-2 human antibody phage display library. 100 nM, 50 nM, 5 nM and 0.5 nM of biotinylated RBD was used for selection rounds 1 to 4. Phage titres used for selection were reduced to 1×10¹¹ for rounds 2 and 3 and 1×10¹⁰ for round 4.

FIG. 2 shows the results of a polyclonal phage ELISA performed with phage pools from selection rounds 1 to 4 using 100 nM of biotinylated SARS-CoV-1 RBD, biotinylated SARS-CoV-2 RBD or Strepavidin.

FIG. 3 shows the results of affinity binding assays (Global fit) performed for two candidate antibodies (A) O4C12 and (B) O4G1, identified in the phage display selection usng the Garvan-2 human antibody phage display library.

FIG. 4 shows the results of phage display off-rate selection for affinity matured antibodies against biotinylated SARS-CoV-2 RBD over 4 rounds using the affinity matured libraries developed from antibodies (A) O4C12 and (B) O4G1.

FIG. 5 shows the results of a polyclonal phage ELISA performed with phage pools from selection rounds 1 to 4 using 50nM biotinylated SARS-CoV-1 RBD, biotinylated SARS-CoV-2 RBD, Streptavidin, Neutravidin and no antigen (empty well).

FIG. 6 shows the results of affinity binding assays (Global fit) performed for four antibodies matured from O4C12: (A) C12K-A10, (B) C12K-B12, (C) C12K-D12 and (D) C 12K-G10.

FIG. 7 shows the results of affinity binding assays (Global fit) performed for two antibodies matured from O4G1: (A) G1K-C2 and (B) G1K-C4.

FIG. 8 shows the results of epitope mapping performed for (A) C12K-A10, (B) C12K-B12, and (C) G1K-C2 against wild-type and mutant RBD of the SARS-CoV-2 S protein.

FIG. 9 shows structural soluble SARS-CoV-2 S protein RBD (SEQ ID NO: 1) in complex with a Fab comprising a G1K-C2 Fab heavy chain (SEQ ID NO: 15) and G1K-C2 light chain (SEQ ID NO: 16), as solved by X-ray crystallography.

FIG. 10 shows the contact interface of the SARS-CoV-2 S protein RBD (SEQ ID NO: 1) with the Fab designated G1K-C2 (V_{H} set forth in SEQ ID NO: 15 and V_{L} set forth in SEQ ID NO: 16).

FIG. 11 shows structural soluble SARS-CoV-2 S protein RBD (SEQ ID NO: 1) in complex with a Fab comprising C12K-B12 Fab heavy chain (SEQ ID NO: 9) and C12K-B12 light chain (SEQ ID NO: 10), as solved by X-ray crystallography.

FIG. 12 shows the contact interface of the SARS-CoV-2 S protein RBD (SEQ ID NO: 1) with the Fab designated C12K-B12 (V_{H} set forth in SEQ ID NO: 9 and V_{L} set forth in SEQ ID NO: 10).

### KEY TO THE SEQUENCE LISTING

SEQ ID NO:1 Amino acid sequence corresponding to the RBD of the SARS-CoV-2 S protein.
SEQ ID NO:2 Amino acid sequence corresponding to the RBD of the SARS-CoV-1 S protein.
SEQ ID NO:3 Amino acid sequence for heavy chain variable domain of antibody designated O4C12.
SEQ ID NO:4 Amino acid sequence for light chain variable domain of antibody designated O4C12.
SEQ ID NO:5 Amino acid sequence for heavy chain variable domain of antibody designated O4G1.
SEQ ID NO:6 Amino acid sequence for light chain variable domain of antibody designated O4G1.
SEQ ID NO:7 Amino acid sequence for heavy chain variable domain of antibody designated C12K-A10.
SEQ ID NO:8 Amino acid sequence for light chain variable domain of antibody designated C12K-A10.
SEQ ID NO:9 Amino acid sequence for heavy chain variable domain of antibody designated C12K-B12.
SEQ ID NO:10 Amino acid sequence for light chain variable domain of antibody designated C12K-B12.
SEQ ID NO:11 Amino acid sequence for heavy chain variable domain of antibody designated C12K-D12.
SEQ ID NO:12 Amino acid sequence for light chain variable domain of antibody designated C12K-D12.
SEQ ID NO:13 Amino acid sequence for heavy chain variable domain of antibody designated C12K-G10.
SEQ ID NO:14 Amino acid sequence for light chain variable domain of antibody designated C12K-G10.
SEQ ID NO:15 Amino acid sequence for heavy chain variable domain of antibody designated G1K-C2.
SEQ ID NO:16 Amino acid sequence for light chain variable domain of antibody designated G1K-C2.
SEQ ID NO:17 Amino acid sequence for heavy chain variable domain of antibody designated G1K-C4.
SEQ ID NO:18 Amino acid sequence for light chain variable domain of antibody designated G1K-C4.
SEQ ID NO:19 Amino acid sequence for heavy chain variable domain CDR1 of antibodies designated O4C12, C12K-A10, C12K-B12, C12K-D12 and C12K-G10.
SEQ ID NO:20 Amino acid sequence for heavy chain variable domain CDR2 of antibodies designated O4C12, C12K-A10, C12K-B12, C12K-D12 and C12K-G10.
SEQ ID NO:21 Amino acid sequence for heavy chain variable domain CDR3 of antibodies designated O4C12 and C12K-A10.
SEQ ID NO:22 Amino acid sequence for light chain variable domain CDR1 of antibodies designated O4C12 and C12K-D12.
SEQ ID NO:23 Amino acid sequence for light chain variable domain CDR2 of antibodies designated O4C12, C12K-A10, C12K-B12 and C12K-D12.
SEQ ID NO:24 Amino acid sequence for light chain variable domain CDR3 of antibodies designated O4C12, C12K-B12, C12K-D12 and C12K-G10.
SEQ ID NO:25 Amino acid sequence for heavy chain variable domain CDR1 of antibodies designated O4G1, G1K-C2 and G1K-C4.
SEQ ID NO:26 Amino acid sequence for heavy chain variable domain CDR2 of antibodies designated O4G1, G1K-C2 and G1K-C4.
SEQ ID NO:27 Amino acid sequence for heavy chain variable domain CDR3 of antibodies designated O4G1, G1K-C2 and G1K-C4.
SEQ ID NO:28 Amino acid sequence for light chain variable domain CDR1 of antibodies designated O4G1 and G1K-C2.
SEQ ID NO:29 Amino acid sequence for light chain variable domain CDR2 of antibodies designated O4G1, G1K-C2 and G1K-C4.
SEQ ID NO:30 Amino acid sequence for light chain variable domain CDR3 of antibodies designated O4G1 and G1K-C4.
SEQ ID NO:31 Amino acid sequence for light chain variable domain CDR1 of antibody designated C12K-Al O.
SEQ ID NO:32 Amino acid sequence for light chain variable domain CDR3 of antibody designated C12K-A10.
SEQ ID NO:33 Amino acid sequence for heavy chain variable domain CDR3 of antibody designated C12K-B12.
SEQ ID NO:34 Amino acid sequence for light chain variable domain CDR1 of antibody designated C12K-B12.
SEQ ID NO:35 Amino acid sequence for heavy chain variable domain CDR3 of antibody designated C12K-D12.
SEQ ID NO:36 Amino acid sequence for heavy chain variable domain CDR3 of antibody designated C12K-G1 O.
SEQ ID NO:37 Amino acid sequence for light chain variable domain CDR1 of antibody designated C12K-G10.
SEQ ID NO:38 Amino acid sequence for light chain variable domain CDR2 of antibody designated C12K-G1 O.
SEQ ID NO:39 Amino acid sequence for light chain variable domain CDR3 of antibody designated G1K-C2.
SEQ ID NO:40 Amino acid sequence for light chain variable domain CDR1 of antibody designated G1K-C4.
SEQ ID NO:41 Amino acid sequence for Bat-RaTG13.
SEQ ID NO:42 Amino acid sequence for Pangolin CoV.

### DETAILED DESCRIPTION OF THE INVENTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the term “severe acute respiratory syndrome coronavirus 2 (SARS-CoV-2)” also known as “2019 novel coronavirus (2019-nCoV)” and “human coronavirus 2019 (HCoV-19 or hCoV-19)” will be understood to refer to a strain of coronavirus that causes coronavirus disease 2019 (COVID-19).

An “antigen-binding protein” as used herein shall be understood to mean a protein comprising an antigen binding region that is capable of specifically binding to one or a few closely related antigens. An exemplary function may be binding to a binding partner. Exemplary antigen-binding proteins include antibodies and antigen binding fragments thereof.

The skilled artisan will be aware that an “antibody” is generally considered to be a protein that comprises a variable region made up of a plurality of immunoglobulin chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ϵ, γ, or μ. Antibodies can be of any type (e.g., IgG, IgE, IgM, IgD, IgA, and IgY), class (e.g., IgG₁, IgG₁, IgG₁, IgG₁, IgA₁ and IgA₁) or subclass. The term “antibody” also encompasses humanized antibodies, de-immunized antibodies, non-depleting antibodies, non-activating antibodies, primatized antibodies, human antibodies, synhumanized antibodies and chimeric antibodies. The antigen binding protein is not a nanobody. As used herein, the term “antibody” is also intended to include formats other than full-length, intact or whole antibody molecules, such as Fab, F(ab′)₂, and Fv which are capable of binding the epitopic determinant. These formats may be referred to as antibody “fragments”. These antibody formats retain some ability to selectively bind to the SARS-CoV-2 S protein RBD, examples of which include, but are not limited to, the following:
(1) Fab, the fragment which contains a monovalent binding fragment of an antibody molecule and which can be produced by digestion of whole antibody with the enzyme papain to yield an intact light chain and a portion of one heavy chain;
(2) Fab′, the fragment of an antibody molecule which can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab)₂ is a dimer of two Fab′ fragments held together by two disulfide bonds;
(4) Fv, defined as a genetically engineered fragment containing the variable region of the light chain and the variable region of the heavy chain expressed as two chains;
(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific (see, for example, WO 94/07921 and WO 98/44001); and
(6) Single domain antibody, typically a variable heavy domain devoid of a light chain.

Accordingly, an antibody in accordance with the present disclosure includes separate heavy chains, light chains, Fab, Fab′, F(ab′)₂, Fc, a variable light domain devoid of any heavy chain, a variable heavy domain devoid of a light chain and Fv. Such fragments can be produced by recombinant DNA techniques, or by enzymatic or chemical separation of intact immunoglobulins.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof. In some cases, the intact antibody may have one or more effector functions.

The antibody disclosed herein may be a humanized antibody. The term “humanized antibody”, as used herein, refers to an antibody derived from a non-human antibody, typically murine, that retains or substantially retains the antigen-binding properties of the parent antibody but which is less immunogenic in humans.

The antibody disclosed herein may be a non-depleting antibody. The term “non-depleting antibody”, as used herein, refers to an antibody that binds to its target but does not recruit the immune system's effector functions which effect target cell lysis. The immune system's effector functions are dependent on interactions of the Fc-domain with C1q, the first component of the complement cascade, and/or receptors (FcR). Complement-dependent cytotoxicity (CDC) is initiated by multiple Fc-domains interacting with C1q, which can ultimately result in lysis of target cells through the formation of the membrane attack complex (MAC). Additionally, cells of the immune system, such as granulocytes, macrophages, and NK cells, may interact via FcRs with mAbs bound to target cells. Lysis of target cells is triggered via antibody-dependent cell mediated cytotoxicity (ADCC) or phagocytosis. Non-depleting antibodies include antibody fragments without an Fc domain, including for example, monovalent (e.g., Fab, scFv, nanobodies and dAbs), bivalent (e.g., F(ab′)₂ and diabodies) and multivalent (e.g., triabodies and pentabodies) formats. In addition, non-depleting antibodies include antibodies that have been modified to remove effector functions without impacting pharmokinetics, for example, amino acid residues in the Fc-domain that play a dominant role in interaction with C1q and FcRs could be modified, or the N-linked glycosylation site in the C_{H}2 domain could be removed. As a skilled person is aware, the chances of engineering a non-depleting antibody are linked to the constant region used to produce the antibody. An IgG3 constant region is more likely to produce a depleting antibody than an IgG1 constant region which in turn is more likely to produce a depleting antibody than an IgG2 constant region, whereas an IgG4 constant region will generally mean that the antibody is non-depleting. A skilled person would also understand that modifications to a constant region could convert a depleting antibody into a non-depleting antibody and vice versa.

The antibody disclosed herein may be a non-activating antibody. As used herein, a “non-activating antibody” refers to antibodies that bind cell surface receptors and negate or block the action of endogenous ligands.

As used herein, “variable region” refers to the portions of the light and/or heavy chains of an antibody as defined herein that is capable of specifically binding to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain. The amino acid positions assigned to CDRs and FRs can be defined according to Kabat (1987 and 1991, supra) or other numbering systems in the performance of methods according to the present disclosure, e.g., the hypervariable loop numbering system of Clothia and Lesk (1987 and/or 1989, supra and/or Al-Lazikani et al., 1997, supra).

As used herein, the term “complementarity determining regions” (syn. CDRs; i.e., CDR1, CDR2, and CDR3) refers to the amino acid residues of an antibody variable domain that form loops between the FRs the sequence of which vary between antibodies. Some or all of the CDRs confer the ability to bind antigen on the antibody. Each variable domain typically has three CDR regions identified as CDR1, CDR2 and CDR3. Each complementarity determining region may comprise amino acid residues from a “complementarity determining region” as defined by Kabat et al., (1991) and/or those residues from a “hypervariable loop” Chothia and Lesk (1987), or any other known numbering technique or combination thereof, including the IMGT numbering system (Le Franc et al., 2003).

“Framework regions” (hereinafter FRs) are those variable domain residues other than the CDR residues.

The term “constant region” or “fragment crystalizable” or “Fc” or “Fc region” or “Fc portion” (which can be used interchangeably herein) as used herein, refers to a portion of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ϵ, γ, or μ. Furthermore, heavy chains of various subclasses (such as the IgG subclasses of heavy chains) are responsible for different effector functions and thus, by choosing the desired heavy chain constant region, proteins with desired effector function can be produced. Preferably, the constant regions of the antibodies of the disclosure are derived from human immunoglobulins. Exemplary heavy chain constant regions are gamma 1 (IgG1), gamma 2 (IgG2), gamma 3 (IgG3), gamma 4 (IgG4), or hybrids thereof The light chain constant region can be of the kappa or lambda type, preferably of the kappa type.

As used herein, the term “Fv” shall be taken to mean any protein, whether comprised of multiple polypeptides or a single polypeptide, in which a V_{L} and a V_{H} associate and form a complex capable of specifically binding to an antigen. The V_{H} and the V_{L} which form the antigen binding domain can be in a single polypeptide chain or in different polypeptide chains. Furthermore, an Fv of the disclosure (as well as any protein of the disclosure) may have multiple antigen binding domains which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, αand δ heavy chains comprises two constant domains.

As will be appreciated by the person skilled in the art, the term “residue” as used herein refers to an amino acid residue. Thus, the word “residue” may be used interchangeably with the term “amino acid”.

The term “recombinant” in the context of an antibody refers to the antibody when produced by a cell, or in a cell-free expression system, in an altered amount or at an altered rate compared to its native state. In one embodiment, the cell is a cell that does not naturally produce the antibody or immunoglobulin chain. However, the cell may be a cell which comprises a non-endogenous gene that causes an altered, preferably increased, amount of the polypeptide to be produced. A recombinant antibody of the disclosure includes polypeptides which have not been separated from other components of the transgenic (recombinant) cell, or cell-free expression system, in which it is produced, and an antibody produced in such cells or cell-free systems which are subsequently purified away from at least some other components.

The antibody disclosed herein may specifically bind to coronavirus S protein RBD, such as SARS-CoV-2 S protein RBD. The RBD is a region within the S protein. For example, in SARS-CoV-2, the RBD corresponding to residues 319-541 of the full length S protein. The sequence of the RBD of SARS-CoV-2 S protein is set forth in SEQ ID NO: 1. The sequence of the RBD of the SARS-CoV-1 S protein is set forth in SEQ ID NO: 2. As used herein, the term “specifically binds” shall be taken to mean a protein reacts or associates more frequently, more rapidly, with greater duration and/or with greater affinity with coronavirus S protein RBD or a specified epitope thereof than it does with alternative antigens or epitopes. As such, “specific binding” does not necessarily require exclusive binding or non-detectable binding of another antigen. The term specifically binds” is used interchangeably with “selectively binds” herein.

As used herein, the term “epitope” (syn. “antigenic determinant”) shall be understood to mean a region of coronavirus S protein RBD, such as a region of SARS-CoV-2 S protein RBD set forth in SEQ ID NO: 1, to which a protein comprising an antibody variable region bind. This term is not necessarily limited to the specific residues or structure to which the protein makes contact. For example, this term includes the region spanning amino acids contacted by the protein and/or at least 5-10 or 2-5 or 1-3 amino acids outside of this region. In some examples, the epitope is a linear series amino acids. However, the epitope is not restricted to only amino acid side-chains. For example, an antigen binding protein described herein binds to an epitope comprising residue 150 of the sequence set forth in SEQ ID NO: 1 (which corresponds to the RBD of the SARS-CoV-2 S protein). For example, an antigen binding protein described herein binds to residue 150I defined relative to the sequence set forth in SEQ ID NO: 1.

The term “binds to an epitope” means that an antibody binds to amino acids within the sequence of the recited epitope. This term does not mean that the antibody binds to each and every amino acid recited, only that one or more of the recited amino acids are necessary for antibody binding.

By “overlapping” in the context of two epitopes shall be taken to mean that two epitopes share a sufficient number of amino acid residues to permit an antibody that binds to one epitope to competitively inhibit the binding of an antibody that binds to the other epitope. For example, the two epitopes share at least 1 or 2 or 3 or 4 or 5 or 6 or more amino acids.

As used herein, the term “neutralise” shall be taken to mean that an antigen binding protein of the disclosure is capable of reducing or preventing infection of mammalian cells. Methods for determining neutralization are known in the art and/or described herein.

The term “competitively inhibits” shall be understood to mean that an antigen-binding protein of the disclosure reduces or prevents binding of a recited antibody to SARS-CoV-2 S protein RBD. This may be due to the antigen-binding protein of the disclosure and recited antibody binding to the same or an overlapping epitope. For example, the antigen-binding protein of the disclosure and recited antibody may both bind to an epitope comprising a isoleucine (I) at position 150 relative to the amino acid sequence set forth in SEQ ID NO: 1. It will be apparent from the foregoing that the antigen-binding protein need not completely inhibit binding of the recited antibody, rather it need only reduce binding by a statistically significant amount, for example, by at least about 10% or 20% or 30% or 40% or 50% or 60% or 70% or 80% or 90% or 95%. Preferably, the antigen-binding protein of the disclosure reduces binding of the recited antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the recited antibody may be exposed to SARS-CoV-2 S protein RBD either in the presence or absence of the antigen-binding protein of the disclosure. If less of the recited antibody binds in the presence of the antigen-binding protein than in the absence of the antigen-binding protein, then the antigen-binding protein is considered to competitively inhibit binding of the recited antibody.

As used herein, the term “overlapping” in the context of two epitopes shall be taken to mean that two epitopes share a sufficient number of amino acid residues to permit an antigen-binding protein (or antibody) that binds to one epitope to competitively inhibit the binding of another antigen-binding protein (or antibody) that binds to the other epitope. For example, the “overlapping” epitopes share at least 1 or 2 or 3 or 4 or 5 or 6 or 7 or 8 or 9 or 20 amino acids. The overlapping epitope shall include position 150, numbered relative to the CoV S protein RBD sequence set forth in SEQ ID NO: 1.

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease or condition (e.g., respiratory infection with SARS-CoV-2 or COVID) are mitigated or eliminated.

As used herein, the terms “preventing”, “prevent” or “prevention” or variations thereof, refers to the provision of prophylaxis with respect to occurrence or recurrence of a disease in an individual. An individual may be predisposed to or at risk of developing the disease or disease relapse but has not yet been diagnosed with the disease or the relapse. The term prevention does not require absolute prevention but includes inhibiting the progression of the disease to some extent.

As used herein, a subject “at risk” of being infected with coronavirus (e.g., such as SARS-CoV-2) and/or developing COVID-19 may or may not have detectable symptoms of infection, and may or may not have displayed detectable symptoms of an infection prior to the treatment according to the present disclosure. “At risk” denotes that a subject has one or more risk factors, which are measurable parameters that correlate with increased susceptibility to infection and development of COVID, as known in the art and/or described herein.

An “effective amount” refers to at least an amount effective, at dosages and for periods of time necessary, to achieve the desired therapeutic or prophylactic result. An effective amount can be provided in one or more administrations. In some examples of the present disclosure, the term “effective amount” is meant an amount necessary to effect treatment of a disease or condition as hereinbefore described. The effective amount may vary according to the disease or condition to be treated and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period.

A “therapeutically effective amount” is at least the minimum concentration required to effect a measurable improvement of a particular disease (e.g., respiratory infection with a coronavirus, such as SARS-CoV-2, or COVID-19). A therapeutically effective amount herein may vary according to factors such as the disease state, age, sex, and weight of the patient, and the ability of the protein to elicit a desired response in the individual. A therapeutically effective amount is also one in which any toxic or detrimental effects of the protein are outweighed by the therapeutically beneficial effects.

A “prophylactically effective amount” refers to an amount effective, at the dosages and for periods of time necessary, to achieve the desired prophylactic result. Typically but not necessarily, since a prophylactic dose is used in mammals prior to or at an earlier stage of disease, a prophylactically effective amount may be less than a therapeutically effective amount.

The term “effective concentration 50%” (abbreviated as “EC₅₀”) represents the concentration of an antigen binding protein, such as an antibody, of the disclosure that is required for 50% of a given effect of the molecule the antibody targets (e.g. neutralisation or prevention of infection of a mammalian cell with a coronavirus, such as SARS-CoV-2). It will be understood by one in the art that a lower EC₅₀ value corresponds to a more potent antibody.

The term “half maximum inhibitory concentration” (abbreviated as “IC₅₀”) represents the concentration of an antigen binding protein, such as an antibody, of the disclosure that is required to achieve 50% neutralisation or prevention of infection of a mammalian cell with coronavirus (e.g., such as SARS-CoV-2). It will be understood by one in the art that a lower IC₅₀ value corresponds to a more potent antibody.

As used herein, the term “subject” shall be taken to mean a mammal. Exemplary subjects include but are not limited to humans and non-human primates. For example, the subject is a human.

The term “expression construct” is to be taken in its broadest context and includes a nucleic acid comprising one or more promoter sequences operably linked with one or more nucleic acids as described herein.

The term “expression vector” refers to a nucleic acid comprising an expression construct that is additionally capable of maintaining and or replicating nucleic acid in an expressible format. For example, an expression vector may comprise a plasmid, bacteriophage, phagemid, cosmid, virus sub-genomic or genomic fragment. Selection of appropriate vectors is within the knowledge of those having skill in the art.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter. A promoter can be operably linked to numerous nucleic acids, e.g., through an internal ribosome entry site.

### Antigen-Binding Proteins Comprising Antibody Variable Regions

An antigen-binding protein of the disclosure comprises an antibody variable region which binds to an epitope of the CoV S protein RBD comprising at least residue 1501 numbered relative to the amino acid sequence set forth in SEQ ID NO: 1. Preferably, the antibody variable region bind to residue 1501 within the epitope of the CoV S protein RBD. In particular, antigen-binding proteins described herein have an affinity for binding to SARS CoV-2 S protein RBD.

SARS-CoV-2 is a member of the Coronaviridae family of enveloped, positive-sense single-stranded RNA viruses. The SARS-CoV-2 protein comprises four structural proteins: spike (S), membrane (M), nucleocapsid (N) and envelope (E).

The S protein is responsible for recognizing the target angiotensin converting enzyme 2 (ACE2) receptor and mediating fusion of the virus and the target cell membrane, which is considered as key to the infection process. The S protein is a large type I transmembrane protein that is highly glycosylated. It contains two subunits, S1 and S2. There are two important domains in S1 subunits, known as the N-Terminal Domain (NTD) and the Receptor Binding Domain (RBD), which is responsible for binding to ACE2. S2 has three domains called Heptad Repeat (HR), Central Helix (CH), and Connector Domain (CD) respectively. Additionally, there is a furin cleavage site at S1/S2.

The S protein protrudes from the viral surface as a homotrimer with two different conformations, pre-fusion and post-fusion. It is the trimeric assembly of the S protein on the virion surface that gives it the distinctive “corona” or crown-like appearance. The binding of the S protein RBD to ACE2 triggers the structural change from pre- to post-fusion, resulting in dissociation of the S1 and S2 subunits and transformation of the S2 subunit into a highly stable post-fusion conformation.

The antigen-binding proteins of the disclosure bind to CoV S protein RBD at an epitope comprising at least residue 150 numbered relative to the amino acid sequence set forth in SEQ ID NO: 1. In particular, the antigen-binding proteins bind to CoV-2 S protein RBD at an epitope comprising an isoleucine (I) at position 150 numbered relative to the amino acid sequence set forth in SEQ ID NO: 1. By binding to the CoV-2 S protein RBD, the antigen-binding proteins are capable of neutralising infection of a mammalian cell with CoV (e.g., including SARS-CoV-2 and SARS-CoV-1).

Exemplary antigen-binding proteins identified/produced by the inventors are described in Table 1.

TABLE 1
Sequences of exemplary antigen-binding proteins

| | Binding protein ID | V_{H} | V_{L} |
|---|---|---|---|
| 1 | O4C12 | SEQ ID NO: 3 | SEQ ID NO: 4 |
| 2 | C12K-A10 | SEQ ID NO: 7 | SEQ ID NO: 8 |
| 3 | C12K-B12 | SEQ ID NO: 9 | SEQ ID NO: 10 |
| 4 | C12K-D12 | SEQ ID NO: 11 | SEQ ID NO: 12 |
| 5 | C12K-G10 | SEQ ID NO: 13 | SEQ ID NO: 14 |
| 6 | O4G1 | SEQ ID NO: 5 | SEQ ID NO: 6 |
| 7 | G1K-C2 | SEQ ID NO: 15 | SEQ ID NO: 16 |
| 8 | G1K-C4 | SEQ ID NO: 17 | SEQ ID NO: 18 |

Antibodies and antigen-binding fragments thereof which compete with an antigen-binding proteins presented in Table 1 are also contemplated, as described herein. Methods of testing competitive binding of antibodies are known in the art.

The antigen-binding proteins of the disclosure may comprise the CDRs of an antigen-binding proteins presented in Table 1, as summarised in Table 2.

TABLE 2
CDRs of exemplary antigen-binding proteins

| | V_{H} | | | V_{L} | | |
|---|---|---|---|---|---|---|
| Binding | CDR1 | CDR2 | CDR3 | CDR1 | CDR2 | CDR3 |
| protein | (SEQ ID NO) | (SEQ ID NO) | (SEQ ID NO) | (SEQ ID NO) | (SEQ ID NO) | (SEQ ID NO) |
| O4C12 | 19 | 20 | 21 | 22 | 23 | 24 |
| C12K-A10 | 19 | 20 | 21 | 31 | 23 | 32 |
| C12K-B12 | 19 | 20 | 33 | 34 | 23 | 24 |
| C12K-D12 | 19 | 20 | 35 | 22 | 23 | 24 |
| C12K-G10 | 19 | 20 | 36 | 37 | 38 | 24 |
| O4G1 | 25 | 26 | 27 | 28 | 29 | 30 |
| G1K-C2 | 25 | 26 | 27 | 28 | 29 | 39 |
| G1K-C4 | 25 | 26 | 27 | 40 | 29 | 30 |

In one example, an antigen-binding protein of the disclosure comprises the CDRs of the antigen binding protein designated O4C12 in Table 1. For example, the antigen-binding protein may comprise an antibody variable region comprising a V_{H} comprising CDR1, CDR2 and CDR3 sequences set forth in SEQ ID NOs: 19, 20 and 21 respectively, and a V_{L} comprising CDR1, CDR2 and CDR3 sequences set forth in SEQ ID NOs: 22, 23 and 24 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 3 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 4 provided that the V_{L} comprises a CDR1 set forth in SEQ ID NO: 22, a CDR2 set forth in SEQ ID NO: 23 and a CDR3 set forth in SEQ ID NO: 24.

In one example, an antigen-binding protein of the disclosure comprises the CDRs of the antigen binding protein designated C12K-A10 in Table 1. For example, the antigen-binding protein may comprise an antibody variable region comprising a V_{H} comprising CDR1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 31, 23 and 32 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 7 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 8 provided that the V_{L} comprises a CDR1 set forth in SEQ ID NO: 31, a CDR2 set forth in SEQ ID NO: 23 and a CDR3 set forth in SEQ ID NO: 32.

In one example, an antigen-binding protein of the disclosure comprises the CDRs of the antigen binding protein designated C12K-B12 in Table 1. For example, the antigen-binding protein may comprise an antibody variable region comprising a V_{H} comprising CDR1, CDR2 and CDR3 comprising the sequences set forth in SEQ ID NOs: 19, 20 and 33 respectively, and a V_{L} comprising CDR1, CDR2 and CDR3 comprising the sequences set forth in SEQ ID NOs: 34, 23 and 24 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 9 provided that the V_{H} comprises a CDR1 set forth in SEQ ID NO: 19, a CDR2 set forth in SEQ ID NO: 20 and a CDR3 set forth in SEQ ID NO: 33, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 10 provided that the V_{L} comprises a CDR1 set forth in SEQ ID NO: 33, a CDR2 set forth in SEQ ID NO: 23 and a CDR3 set forth in SEQ ID NO: 24.

In one example, an antigen-binding protein of the disclosure comprises the CDRs of the antigen binding protein designated C12K-D12 in Table 1. For example, the antigen-binding protein may comprise an antibody variable region comprising a V_{H} comprising CDR1, CDR2 and CDR3 comprising the sequences set forth in SEQ ID NOs: 19, 20 and 35 respectively, and a V_{L} comprising CDR1, CDR2 and CDR3 comprising the sequences set forth in SEQ ID NOs: 22, 23 and 24 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 11 provided that the V_{H} comprises a CDR1 set forth in SEQ ID NO: 19, a CDR2 set forth in SEQ ID NO: 20 and a CDR3 set forth in SEQ ID NO: 35, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 12 provided that the V_{L} comprises a CDR1 set forth in SEQ ID NO: 22, a CDR2 set forth in SEQ ID NO: 23 and a CDR3 set forth in SEQ ID NO: 24.

In one example, an antigen-binding protein of the disclosure comprises the CDRs of the antigen binding protein designated C12K-G10 in Table 1. For example, the antigen-binding protein may comprise an antibody variable region comprising a V_{H} comprising CDR1, CDR2 and CDR3 comprising the sequences set forth in SEQ ID NOs: 19, 20 and 36 respectively, and a V_{L} comprising CDR1, CDR2 and CDR3 comprising the sequences set forth in SEQ ID NOs: 37, 38 and 24 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical)to the sequence set forth in SEQ ID NO: 13 provided that the V_{H} comprises a CDR1 set forth in SEQ ID NO: 19, a CDR2 set forth in SEQ ID NO: 20 and a CDR3 set forth in SEQ ID NO: 36, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 14 provided that the V_{L} comprises a CDR1 set forth in SEQ ID NO: 37, a CDR2 set forth in SEQ ID NO: 38 and a CDR3 set forth in SEQ ID NO: 24.

In one example, an antigen-binding protein of the disclosure comprises the CDRs of the antigen binding protein designated O4G1 in Table 1. For example, the antigen-binding protein may comprise an antibody variable region comprising a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 30 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 5 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 6 provided that the V_{L} comprises a CDR1 set forth in SEQ ID NO: 28, a CDR2 set forth in SEQ ID NO: 29 and a CDR3 set forth in SEQ ID NO: 30.

In one example, an antigen-binding protein of the disclosure comprises the CDRs of the antigen binding protein designated G1K-C2 in Table 1. For example, the antigen-binding protein may comprise an antibody variable region comprising a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 39 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 15 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 16 provided that the V_{L} comprises a CDR1 set forth in SEQ ID NO: 28, a CDR2 set forth in SEQ ID NO: 29 and a CDR3 set forth in SEQ ID NO: 39.

In one example, an antigen-binding protein of the disclosure comprises the CDRs of the antigen binding protein designated G1K-C4 in Table 1. For example, the antigen-binding protein may comprise an antibody variable region comprising a Vx comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 40, 29 and 30 respectively. For example, the antigen binding protein may comprise an antibody variable region comprising a V_{H} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 17 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical (e.g., at least about 95% identical, or at least about 96% identical, or at least about 97% identical, or at least about 98% identical, or at least about 99% identical or 100% identical) to the sequence set forth in SEQ ID NO: 18 provided that the V_{L} comprises a CDR1 set forth in SEQ ID NO: 40, a CDR2 set forth in SEQ ID NO: 29 and a CDR3 set forth in SEQ ID NO: 30.

Exemplary antigen-binding proteins are antibodies. In one example, the antibody is a recombinant antibody. For example, an antibody or antigen-binding protein as described herein may be produced using a standard method, e.g., as is known in the art or briefly described herein.

Monoclonal antibodies are exemplary antibodies contemplated by the present disclosure. The term “monoclonal antibody” or “mAb” or “MAb” refers to a homogeneous antibody population capable of binding to the same antigen(s) and, for example, to the same epitope within the antigen. This term is not intended to be limited with respect to the source of the antibody or the manner in which it is made.

### Deimmunized, Chimeric, Humanized, Synhumanized, Primatized and Human Antigen-Binding Proteins

The antigen-binding proteins of the present disclosure may be a humanized.

The term “humanized”, as used in the context of antigen-binding proteins, shall be understood to refer to an antigen-binding proteins comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized antigen-binding proteins also include antigen-binding proteins in which one or more residues of the human antigen-binding protein are modified by one or more amino acid substitutions and/or one or more FR residues of the antigen-binding human protein are replaced by corresponding non-human residues. Humanized antigen-binding proteins may also comprise residues which are found in neither the human antibody or in the non-human antibody. Any additional regions of the antigen-binding protein (e.g., Fc region) are generally human. Humanization can be performed using a method known in the art, e.g., U.S. Pat. Nos. 5,225,539, 6,054,297, 7,566,771 or 5,585,089. The term “humanized protein” also encompasses a super-humanized antigen-binding protein, e.g., as described in U.S. Pat. No. 7,732,578.

The antigen-binding proteins of the present disclosure may be human antigen-binding proteins. The term “human”, as used in the context of antigen-binding proteins, as used herein refers to antigen-binding proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the antigen-binding protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include an antigen-binding protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

The antigen-binding proteins of the present disclosure may be synhumanized. The term “synhumanized”, as used in the context of antigen-binding proteins, refers to an antigen-binding protein prepared by a method described in W02007/019620. A synhumanized antigen-binding protein includes a variable region of an antibody, wherein the variable region comprises FRs from a New World primate antibody variable region and CDRs from a non-New World primate antibody variable region. For example, a synhumanized antigen-binding protein includes a variable region of an antibody, wherein the variable region comprises FRs from a New World primate antibody variable region and CDRs from a mouse or rat antibody.

The antigen-binding proteins of the present disclosure may be primatized. A “primatized antigen-binding protein” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example an antigen-binding protein of the disclosure is chimeric. The term “chimeric”, as used in the context of antigen-binding proteins, refers to antigen-binding proteins in which an antigen binding domain is from a particular species (e.g., murine, such as mouse or rat) or belonging to a particular antibody class or subclass, while the remainder of the antigen-binding protein is from a protein derived from another species (such as, for example, human or non-human primate) or belonging to another antibody class or subclass. In one example, a chimeric antigen-binding protein is a chimeric antibody comprising a V_{X} and/or a V_{L} from a non-human antibody (e.g., a murine antibody) and the remaining regions of the antibody are from a human antibody. The production of such chimeric proteins is known in the art, and may be achieved by standard means (as described, e.g., in U.S. Pat. Nos. 6,331,415; 5,807,715; 4,816,567 and 4,8163,97).

The present disclosure also contemplates a deimmunized antigen-binding protein, e.g., as described in WO2000/34317 and WO2004/108158. De-immunized antibodies and antigen-binding proteins have one or more epitopes, e.g., B cell epitopes or T cell epitopes removed (i.e., mutated) to thereby reduce the likelihood that a subject will raise an immune response against the antibody or antigen-binding protein.

### Antibody Fragments

Single Chain Fv (scFv) Fragments and dimeric-scFv (di-scFv)

The skilled artisan will be aware that scFvs comprise Vx_{H} and V_{L} regions in a single polypeptide chain. The polypeptide chain further comprises a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favoured linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv (see, for example, Brinkmann et al., 1993).

Alternatively, or in addition, the present disclosure provides a dimeric scFv, i.e., an antigen-binding protein comprising two scFv molecules linked by a non-covalent or covalent linkage, e.g., by a leucine zipper domain (e.g., derived from Fos or Jun) (see, for example, Kruif and Logtenberg, 1996). Alternatively, two scFvs are linked by a peptide linker of sufficient length to permit both scFvs to form and to bind to an antigen, e.g., as described in US20060263367.

For a review of scFv, see Pluckthun (1994).

### Diabodies, Triabodies, Tetrabodies

Exemplary antigen-binding proteins comprising an antibody antigen binding domain are diabodies, triabodies, tetrabodies and higher order protein complexes such as those described in WO98/044001 and WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form an Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

### Minibodies

The skilled artisan will be aware that a minibody comprises the V_{H} and V_{L} domains of an antibody fused to the C_{H}2 and/or C_{H}3 domain of an antibody. Optionally, the minibody comprises a hinge region between the V_{H} and a V_{L}, sometimes this conformation is referred to as a Flex Minibody. A minibody does not comprise a C_{H}1 or a C_{L}. In one example, the V_{H} and V_{L} domains are fused to the hinge region and the C_{H}3 domain of an antibody. At least one of the variable regions of said minibody binds to the S protein RBD in the manner of the disclosure. Exemplary minibodies and methods for their production are described, for example, in WO94/09817.

### Constant Domain Fusions

The present disclosure encompasses antigen-binding proteins comprising a variable region and a constant region or a domain(s) thereof, e.g., Fc, C_{H}2 and/or C_{H}3 domain. The skilled artisan will be aware of the meaning of the terms constant region and constant domain based on the disclosure herein and references discussed herein.

Constant region sequences useful for producing the antigen-binding proteins of the present disclosure may be obtained from a number of different sources. In some examples, the constant region or portion thereof of the protein is derived from a human antibody. Moreover, the constant domain or portion thereof may be derived from any antibody class, including IgM, IgG, IgD, IgA and IgE, and any antibody isotype, including IgG₁, IgG₂, IgG₃ and IgG₄.

A variety of constant region gene sequences are available in the form of publicly accessible deposits or the sequence thereof is available from publicly available databases. Constant regions can be selected having a particular effector function (or lacking a particular effector function) or with a particular modification to reduce immunogenicity.

The present disclosure also contemplates antigen-binding proteins comprising mutant constant regions or domains, e.g., as described in U.S. Pat. Nos. 7,217,797; 7,217,798; or US20090041770 (having increased half-life) or US2005037000 (increased ADCC).

### Protein Production

In one example, an antigen-binding protein of the disclosure is produced by culturing a cell line under conditions sufficient to produce the antigen-binding protein, e.g., as described herein and/or as is known in the art.

### Recombinant Expression

In the case of a recombinant protein, a nucleic acid encoding same is placed into one or more expression construct, e.g., expression vector(s), which is/are then transfected into host cells, such as cells that can produce a disulphide bridge or bond, such as E. coli cells, yeast cells, insect cells, or mammalian cells. Exemplary mammalian cells include simian COS cells, Chinese Hamster Ovary (CHO) cells, or myeloma cells that do not otherwise produce immunoglobulin protein. Molecular cloning techniques to achieve these ends are known in the art and described, for example in Ausubel or Sambrook. A wide variety of cloning and in vitro amplification methods are suitable for the construction of recombinant nucleic acids. Methods of producing recombinant antibodies are also known in the art. See U.S. Pat. Nos. 4,816,567; 7,923,221 and 7,022,500.

Following isolation, the nucleic acid encoding an antigen-binding protein of the disclosure is inserted into an expression construct or replicable vector for further cloning (amplification of the DNA) or for expression in a cell-free system or in cells. For example, the nucleic acid is operably-linked to a promoter,

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably-linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding an antigen-binding protein of the present disclosure (e.g., derived from the amino acid sequences provided herein), an enhancer element, a promoter, and a transcription termination sequence. The skilled artisan will be aware of suitable sequences for expression of a protein. For example, exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters include those active in prokaryotes (e.g., phoA promoter, β-lactamase and lactose promoter systems, alkaline phosphatase, a tryptophan (trp) promoter system, and hybrid promoters such as the tac promoter).

Exemplary promoters active in mammalian cells include cytomegalovirus immediate early promoter (CMV-IE), human elongation factor 1-a promoter (EF1), small nuclear RNA promoters (U1a and U1b), a-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof Examples of useful mammalian host cell lines are monkey kidney CV1 line transformed by SV40 (COS-7, AUSTRALIAN CELL BANK CRL 1651); human embryonic kidney line (293 or 293 cells subcloned for growth in suspension culture; baby hamster kidney cells (BHK, AUSTRALIAN CELL BANK CCL or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHOS promoter, the nmt promoter, the RPR1 promoter, or the TEF1 promoter.

Means for introducing the isolated nucleic acid molecule or a gene construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation, viral transduction (e.g., using a lentivirus) and microparticle bombardment such as by using DNA-coated tungsten or gold particles (Agracetus Inc., WI, USA) amongst others.

The host cells used to produce the antigen-binding proteins of the disclosure may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation of Antigen-Binding Proteins

An antigen-binding protein of the present disclosure can be isolated or purified.

Methods for purifying an antigen-binding protein of the disclosure are known in the art and/or described herein.

When using recombinant techniques, the antigen-binding protein of the disclosure can be produced intracellularly, in the periplasmic space, or directly secreted into the medium. If the antigen-binding protein is produced intracellularly, as a first step, the particulate debris, either host cells or lysed fragments, is removed, for example, by centrifugation or ultrafiltration. Where the protein is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antigen-binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Zola (1997).

The skilled artisan will also be aware that an antigen-binding protein of the disclosure can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. For example, the tag is a hexa-his tag. The resulting antigen-binding protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein.

Alternatively, or in addition a ligand or antibody that binds to a tag is used in an affinity purification method.

### Conjugates

The present disclosure also provides conjugates of antigen-binding proteins described herein according to any example. For example, an antigen-binding protein comprising an antibody variable region is conjugated to a detectable label, a therapeutic compound, a colloid, a toxin, a nucleic acid, a peptide, a protein, a compound that increases the half-life of the protein in a subject and mixtures thereof

As used herein, the term “conjugate” or “conjugated” shall be understood to encompass both indirect and direct binding. For example, direct conjugation includes chemical conjugation, which can be non-covalent or covalent or genetic conjugation (also referred to as “fusion”). In one example, the conjugation is covalent, e.g., a disulphide bond.

As used herein, a “detectable label” is a molecular or atomic tag or marker that generates or can be induced to generate an optical or other signal or product that can be detected visually or by using a suitable detector. Detectable labels are well known in the art and include, for example, a radiolabel, an enzyme, a fluorescent label, a luminescent label, a bioluminescent label, a magnetic label, a prosthetic group, a contrast agent and an ultrasound agent.

In one example, an antigen-binding protein as described herein according to any example may be conjugated or linked to another protein, including another antigen-binding protein of the disclosure or a further protein comprising an antibody variable region, such as an antibody or an antigen-binding protein derived therefrom. Other proteins are not excluded. Additional proteins will be apparent to the skilled artisan and include, for example, an immunomodulator or a half-life extending protein or a peptide or other protein that binds to serum albumin amongst others.

Exemplary serum albumin binding peptides or protein are described in US20060228364 or US20080260757.

The antigen-binding proteins of the present disclosure can be modified to contain additional non-proteinaceous moieties that are known in the art and readily available. For example, the moieties suitable for derivatization of the antigen-binding protein are physiologically acceptable polymer, e.g., a water soluble polymer. Such polymers are useful for increasing stability and/or reducing clearance (e.g., by the kidney) and/or for reducing immunogenicity of an antigen-binding protein of the disclosure. Non-limiting examples of water soluble polymers include, but are not limited to, polyethylene glycol (PEG), polyvinyl alcohol (PVA), or propropylene glycol (PPG).

In one example, an antigen-binding protein as described herein according to any example comprises one or more detectable markers to facilitate detection and/or isolation. For example, the compound comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, or in addition, the antigen-binding protein as described herein according to any example is labelled with, for example, a fluorescent semiconductor nanocrystal (as described, for example, in U.S. Pat. No. 6,306,610).

Alternatively, or in addition, the antigen-binding protein is labelled with, for example, a magnetic or paramagnetic compound, such as, iron, steel, nickel, cobalt, rare earth materials, neodymium-iron-boron, ferrous-chromium-cobalt, nickel-ferrous, cobalt-platinum, or strontium ferrite.

### Assaying Antigen-Binding Proteins of the Disclosure

Antigen-binding proteins of the disclosure may be readily screened for physical and biological activity and/or stability using methods known in the art and/or as described below.

### Binding to a CoV S Protein RBD

It will be apparent to the skilled artisan from the disclosure herein that an antigen-binding protein of the present disclosure binds (or specifically binds) to the CoV S protein RBD (including, but not limited to the RBD of SARS-CoV-2 S protein as set forth in SEQ ID NO: 1). Methods for assessing binding to an antigen-binding protein are known in the art, e.g., as described in Scopes (In: Protein purification: principles and practice, Third Edition, Springer Verlag, 1994). Such a method generally involves labelling the antigen-binding protein and contacting it with immobilised compound. Following washing to remove non-specific bound protein, the amount of label and, as a consequence, bound antigen-binding protein is detected. Of course, the antigen-binding protein can be immobilised and the compound that binds to RBD of CoV spike protein labelled. Panning-type assays can also be used. Alternatively, or additionally, surface plasmon resonance assays can be used.

The assays described above can also be used to detect the level of binding of an antigen-binding protein of the present disclosure to RBD of CoV S protein (e.g., such as the RBD of SARS-CoV-2 S protein as set forth in SEQ ID NO: 1). Methods of detecting the level of binding will be apparent to the skilled person and/or described herein. For example, the level of binding is determined using a biosensor.

### Neutralising Assays

Antigen-binding proteins of the disclosure may be screened in vitro for their ability to bind to CoV S protein RBD (e.g., SARS-CoV-2 S protein RBD) and neutralises infection of a mammalian cell. Suitable assays will be apparent to the skilled person and include, for example, a Vero microneutralisation assay, a sVNT assay, or a psuedovirus neutralisation assay (using e.g., HEK-293T cells or HeLa-ACE2 cells).

In one example, the neutralization assay is a Vero microneutralization assay. Briefly, a CoV wild-type virus (e.g., SARS-CoV-2 wildtype virus) is passaged in Vero cells (i.e., the Vero lineage isolated from kidney epithelial cells extracted from an African green monkey). Serial two-fold dilutions of a test antigen-binding protein are incubated with 100 TCID₅₀ (i.e., median tissue culture infectious dose) of CoV for 1 hour and residual virus infectivity is assessed in Vero cells; viral cytopathic effect is read, for example, on day 5. The neutralising antibody titre is calculated using the Reed/Muench method as previously described (Houser et al., 2016; Subbarao et al 2004).

In one example, the neutralization assay is a surrogate neutralization test (sVNT). Briefly, the wells of a plate are coated with hACE2 protein in carbonate-bicarbonate coating buffer (e.g., pH 9.6). HRP-conjugated CoV (e.g., SARS-CoV-2) and HRP-conjugated CoV S protein RBD (e.g., SARS-CoV-2 S protein RBD) pre-incubated with test antigen-binding proteins are added to the hACE2 at different concentrations and incubated, for example, for 1 h at room temperature. Unbound HRP conjugated antigens are removed by washing. Colorimetric signal is developed on the enzymatic reaction of HRP with chromogenic substrate, e.g., 3,3′,5,5′-tetramethylbenzidine (TMB). In one example, the absorbance reading at 450 nm and 570 nm is acquired.

In one example, the neutralisation is a psuedovirus neutralisation assay. Briefly, HIV reporter virus pseudotyped with CoV S protein (e.g., SARS-CoV-2 S protein) is produced by co-transfection of CoV S protein (e.g., SARS-CoV-2 S protein) plasmids together with a viral backbone plasmid (e.g., pDR-NL Aenv FLUC) into e.g., HEK-293T cells. Pseudovirus is harvested post transfection and clarified by filtration. Virus stock titres, reported as Relative Luciferase Units infectious dose (RLU), are calculated by limiting dilution infections in Hela-hACE2 cells measuring luciferase activity as a read-out for viral infection.

### Determining Competitive Binding

Assays for determining an antigen-binding protein that competitively inhibits binding of any one of antibodies designated O4C12, C12K-A1 0, C12K-B12, C12K-D12, C12K-G10, O4G1, G1K-C2 and G1K-C4 (or any other antibody described herein) will be apparent to the skilled artisan. For example, O4C12, C12K-A10, C12K-B12, C12K-D12, C12K-G10, O4G1, G1K-C2 or G1K-C4 is conjugated to a detectable label, e.g., a fluorescent label or a radioactive label. The labelled antibody and the test antigen-binding protein are then mixed and contacted with a CoV S protein RBD (e.g., SARS-CoV-2 S protein RBD) or a region thereof or a cell expressing same. The level of labelled 6G6, 19G2, 30B8, 39E7 or 30E10 is then determined and compared to the level determined when the labelled antibody is contacted with the SARS-CoV-2 S protein RBD, region or cells in the absence of the protein.

If the level of labelled O4C12, C12K-A10, C12K-B12, C12K-D12, C12K-G10, O4G1, G1K-C2 or G1K-C4 is reduced in the presence of the test antigen-binding protein compared to the absence of the test antigen-binding protein, then the test antigen binding protein is considered to competitively inhibit binding of the antibodies designated O4C12, C12K-A10, C12K-B12, C12K-D12, C12K-G10, O4G1, G1K-C2 or G1K-C4 to the CoV S protein RBD (e.g., SARS-CoV-2 S protein RBD) or region thereof.

Optionally, the test antigen-binding protein is conjugated to a different label to the antibody designated O4C12, C12K-A10, C12K-B12, C12K-D12, C12K-G10, O4G1, G1K-C2 or G1K-C4. This alternate labelling permits detection of the level of binding of test antigen-binding protein to CoV S protein RBD (e.g., SARS-CoV-2 S protein RBD) or region thereof or the cell.

In another example, the antigen-binding protein is permitted to bind to CoV S protein RBD (e.g., SARS-CoV-2 S protein RBD) or region thereof or the cell expressing same prior to contacting the CoV S protein RBD (e.g., SARS-CoV-2 S protein RBD) or region thereof or the cell expressing the same with O4C12, C12K-A10, C12K-B12, C12K-D12, C12K-G10, O4G1, G1K-C2 or G1K-C4. A reduction in the amount of bound O4C12, C12K-A10, C12K-B12, C12K-D12, C12K-G10, O4G1, G1K-C2 or G1K-C4 in the presence of the antigen-binding protein compared to in the absence of the antigen binding protein indicates that the antigen-binding protein competitively inhibits O4C12, C12K-A10, C12K-B12, C12K-D12, C12K-G10, O4G1, G1K-C2 or G1K-C4 binding to RBD of CoV S protein (e.g., RBD of SARS-CoV-2 S protein). A reciprocal assay can also be performed using labelled antigen-binding protein and first allowing O4C12, C12K-A10, C12K-B12, C12K-D12, C12K-G10, O4G1, G1K-C2 or G1K-C4 to bind to RBD of CoV S protein (e.g., RBD of SARS-CoV-2 S protein). In this case, a reduced amount of labelled antigen-binding protein bound to SARS-CoV-2 S protein RBD in the presence of O4C12, C12K-A10, C12K-B12, C12K-D12, C12K-G10, O4G1, G1K-C2 or G1K-C4 compared to in the absence of O4C12, C12K-A10, C12K-B12, C12K-D12, C12K-G10, O4G1, G1K-C2 or G1K-C4 indicates that the antigen-binding protein competitively inhibits binding of O4C12, C12K-A10, C12K-B12, C12K-D12, C12K-G10, O4G1, G1K-C2 or G1K-C4 to CoV S protein RBD (e.g., SARS-CoV-2 S protein RBD).

### Epitope Mapping of Antigen-Binding Proteins

Assays for determining binding sites of an antigen-binding protein disclosed herein to one or more epitopes of CoV S protein RBD (e.g., such as SARS-CoV-2 S protein RBD) will be apparent to the skilled artisan. In an example, the antigen-binding protein is permitted to bind to a linear epitope of the CoV S protein RBD (e.g., such as SARS-CoV-2 S protein RBD). For example, an antigen-binding protein described herein may be contacted with an epitope of a CoV S protein RBD (e.g., such as SARS-CoV-2 S protein RBD) and binding determined by a specific assay (e.g. ELISA, Western Blotting, X-ray crystallography, 3D Electron Microscopy, Liquid chromatography-mass spectrometry). In one example, the assay is X-ray crystallography.

### Methods of Use

As discussed herein, the present disclosure provides a method for treating or preventing infection with a CoV in a subject in need thereof, the method comprising administering to the subject the antigen-binding protein, the nucleic acid(s), expression construct(s) or the composition of the disclosure.

In one example, the subject is suffering from a respiratory infection with coronavirus (i.e., the subject is in need of treatment). For example, the respiratory infection is an infection with SARS-CoV-2 (i.e., coronavirus disease 2019 (COVID-19)). In accordance with an example in which the subject is suffering from a respiratory infection with coronavirus, the antigen-binding protein, the nucleic acid(s), expression construct(s) or the composition of the disclosure is administered to the subject in an amount sufficient and effective to reduce the severity of the infection and/or reduce one or more symptoms thereof in the subject.

In one example, the coronavirus which causes the respiratory infection is SARS-CoV-2. In another example, the coronavirus which causes the respiratory infection is SARS-CoV-1. In another example, the coronavirus which causes the respiratory infection is MERS-CoV

In another example, an antigen-binding protein of the present disclosure can be administered to an individual by an appropriate route in combination with (before, simultaneous with, or after) another drug or agent for treating respiratory infection with coronavirus (e.g., such as SARS-CoV-2). For example, the antigen-binding protein of the present disclosure may be administered in combination with an antiviral compound known to be useful for treating or delaying progression of respiratory infection with coronavirus (e.g., such as SARS-CoV-2). For example, the antigen-binding protein of the present disclosure may be administered in combination a further antigen-binding protein (e.g, a further antibody) which targets a different epitope of a coronavirus protein (e.g., such as a different epitope of the RBD of the SARS-CoV-2 S protein).

In one example, the method additionally comprises identifying a subject suffering from a respiratory coronavirus infection. Methods of identifying such a subject will be apparent to the skilled person and/or are described herein. For example, a method of identifying a subject suffering from a respiratory coronavirus infection may comprising detecting the presence or absence of a CoV S protein RBD in a biological sample obtained from the subject. Such a method may comprise:
(i) contacting the sample with an antigen-binding protein of the disclosure; and
(ii) analysing the sample for binding between CoV S protein RBD and the antigen-binding protein. Binding of the antigen-binding protein to the CoV S protein RBD indicates the presence of CoV in the biological sample. The presence or absence of a CoV S protein RBD in the biological sample may then be used to diagnose whether or not the subject is infected with coronavirus. For example, detection of binding of the antigen-binding protein to the CoV S protein RBD in a biological sample obtained from a subject indicates that the subject is positive for CoV infection.

In one example, the method of detecting or diagnosing CoV infection is performed in vitro and the sample is, or has been obtained from, a nasopharyngeal swab, a oropharyngeal swab, a nasal aspirate, a nasal wash, saliva, sputum, tracheal aspirate or bronchoalveolar lavage (BAL).

In another example, the present disclosure provides a method for preventing infection with a CoV in a subject in need thereof (e.g., a subject at risk of developing a respiratory coronavirus infection). The method of preventing infection with a CoV comprises administering to the subject the antigen-binding protein, the nucleic acid(s), expression construct(s) or the composition of the disclosure. The method may prevent infection by SARS-CoV-2, SARS-CoV-1 or MERS-CoV. In one example, the coronavirus is SARS-CoV-2.

A subject is at risk if he or she has a higher risk of developing a respiratory coronavirus (e.g., SARS-CoV-2) infection than a control population. The control population may include one or more subjects selected at random from the general population (e.g., matched by age, gender, race and/or ethnicity) who have not suffered from or have a family history of a respiratory viral infection. A subject can be considered at risk for a complement mediated disorder if a “risk factor” associated with a respiratory viral infection is found to be associated with that subject. A risk factor can include any activity, trait, event or property associated with a given respiratory coronavirus infection, for example, through statistical or epidemiological studies on a population of subjects. A subject can thus be classified as being at risk for a respiratory coronavirus infection even if studies identifying the underlying risk factors did not include the subject specifically.

Non-exhaustive examples of subjects at higher risk of developing a respiratory coronavirus (e.g., SARS-CoV-2) infection include subjects which satisfy one or more of the following: over 70 years of age, immunosuppressed, immune deficient, receiving immunosuppressive therapy, received a bone-marrow transplant in past 12 months, suffering from a blood cancer, receiving treatment for cancer, suffer from chronic kidney failure, suffer from heart disease, suffer from chronic lung disease, suffer from diabetes, suffer from chronic liver disease, and any combination thereof.

In one example, treatment according to the method of the disclosure reduces one or more symptoms of a respiratory coronavirus infection (e.g. SARS-CoV-2 infection, or COVID-19). Similarly, prevention according to the method of the disclosure prevents onset of one or more symptoms of a respiratory coronavirus infection (e.g. SARS-CoV-2 infection, or COVID-19). Accordingly, the methods described herein comprise administering an antigen-binding protein, the nucleic acid(s), expression construct(s), or composition of the disclosure in an amount sufficient to reduce the severity of, or prevent onset of, one or more symptoms of a respiratory coronavirus infection e.g., SARS-CoV-2 infection or COVID-19. Symptoms of a SARS-CoV-2 infection or COVID-19 will be apparent to the skilled person and/or are described herein.

As will be apparent to the skilled person a “reduction” in a symptom of a respiratory coronavirus (e.g., SARS-CoV-2) infection in a subject will be comparative to another subject who also suffers from a respiratory coronavirus (e.g., SARS-CoV-2) infection but who has not received treatment with a method described herein. This does not necessarily require a side-by-side comparison of two subjects. Rather population data can be relied upon. For example, a population of subjects suffering from a respiratory coronavirus (e.g., SARS-CoV-2) infection who have not received treatment with a method described herein (optionally, a population of similar subjects to the treated subject, e.g., age, weight, race) are assessed and the mean values are compared to results of a subject or population of subjects treated with a method described herein.

In one example, performing a method described herein according to any example of the disclosure results in enhancement of a clinical response and/or delayed disease progression.

By “clinical response” is meant an improvement in the symptoms of disease (e.g., COVID-19). The clinical response may be achieved within a certain time frame, for example, within or at about 8 weeks from the start of treatment with, or from the initial administration. Clinical response may also be sustained for a period of time, such as for >24 weeks, or ≥48 weeks.

### Pharmaceutical Compositions

Antigen-binding proteins of the disclosure (syn. active ingredients) are useful for formulations into a pharmaceutical composition for parenteral, topical, oral, or local administration, aerosol administration, or transdermal administration, for prophylactic or for therapeutic treatment. The pharmaceutical compositions can be administered in a variety of unit dosage forms depending upon the method of administration. For example, unit dosage forms suitable for oral administration include powder, tablets, pills, capsules and lozenges.

The pharmaceutical compositions of this disclosure are useful for parenteral administration, such as intravenous administration or subcutaneous administration or administration into a body cavity or lumen of an organ or joint. The compositions for administration will commonly comprise a solution of the antigen-binding protein of the disclosure dissolved in a pharmaceutically acceptable carrier, such as an aqueous carrier. A variety of aqueous carriers can be used, e.g., buffered saline and the like. The compositions may contain pharmaceutically acceptable carriers as required to approximate physiological conditions such as pH adjusting and buffering agents, toxicity adjusting agents and the like, for example, sodium acetate, sodium chloride, potassium chloride, calcium chloride, sodium lactate and the like. The concentration of the antigen-binding protein of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

The antigen-binding protein of the disclosure can be formulated for parenteral administration, e.g., formulated for injection via the intravenous, intramuscular, sub-cutaneous, transdermal, or other such routes, including peristaltic administration and direct instillation into a tumor or disease site (intracavity administration). The preparation of an aqueous composition that contains the compounds of the present disclosure as an active ingredient will be known to those of skill in the art.

Suitable pharmaceutical compositions in accordance with the disclosure will generally include an amount of the antigen-binding protein of the present disclosure admixed with an acceptable pharmaceutical carrier, such as a sterile aqueous solution, to give a range of final concentrations, depending on the intended use. The techniques of preparation are generally known in the art as exemplified by Remington's Pharmaceutical Sciences, 16th Ed. Mack Publishing Company, 1980.

### Dosage and Administration

Upon formulation, antigen-binding proteins of the present disclosure will be administered in a manner compatible with the dosage formulation and in such amount as is therapeutically/prophylactically effective.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the antigen-binding protein is administered at an initial (or loading) dose which is higher than subsequent (maintenance doses). For example, the antigen-binding protein is administered at an initial dose of between about 1 mg/kg to about 30 mg/kg. The antigen-binding protein is then administered at a maintenance dose of between about to about 1 mg/kg. For example, the maintenance doses may be administered every 7-35 days, such as, every 14 or 21 or 28 days. In another example, the maintenance dose may be administered every 3-12 months, such as about every 3 months, or about every 6 months or about every 12 months.

In some examples, treatment or prevention may comprise administering multiple doses of the antigen-binding protein of the disclosure to the subject at different time points. For example, a first and a second and/or subsequent dose may be administered at defined intervals, for example about 4-6 weeks apart, or about 6-12 weeks apart, or about 12-18 weeks apart or about 18-24 weeks apart.

In some examples, a dose escalation regime is used, in which an antigen-binding protein is initially administered at a lower dose than used in subsequent doses. This dosage regime is useful in the case of subject's initially suffering adverse events.

In the case of a subject that is not adequately responding to treatment, multiple doses in a week may be administered. Alternatively, or in addition, increasing doses may be administered.

A subject may be retreated with the antigen-binding protein by being given more than one exposure or set of doses, such as at least about two exposures of the antigen-binding protein, for example, from about 2 to 60 exposures, and more particularly about 2 to 40 exposures, most particularly, about 2 to 20 exposures.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

### Kits

The present disclosure also provides kits containing an antigen-binding protein, a nucleic acid, expression construct or composition of the disclosure useful for treating or preventing infection with CoV (e.g., a SARS-CoV-2 infection) in a subject as described above.

In one example, the kit comprises (a) a container comprising an antigen-binding protein, a nucleic acid, expression construct or composition of the disclosure packaged with instructions for use in treating or preventing infection with CoV (e.g., a SARS-CoV-2 infection) in a subject according to the method described herein. The kit may further comprise a delivery system. In accordance with this example of the disclosure, the package insert (with instructions for use) is on or associated with the container. Suitable containers include, for example, bottles, vials, syringes, etc. Accordingly, the container may serve as the delivery system. The containers may be formed from a variety of materials such as glass or plastic. The container holds or contains a composition that is effective for treating or preventing infection with CoV (e.g., SARS-CoV-2 infection) and may have a sterile access port (for example, the container may be an intravenous solution bag or a vial having a stopper pierceable by a hypodermic injection needle). At least one active agent in the composition is the antigen-binding protein or a nucleic acid encoding same (e.g., in the case of a mRNA-based vaccine). The label or package insert indicates that the composition is used for treating a subject eligible for treatment, e.g., one having or at risk of developing a respiratory coronavirus infection (e.g., SARS-CoV-2 infection) with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

In another example, the present disclosure also provides a kit comprising an antigen-binding protein of the disclosure packaged with instructions for use in detecting the presence or absence of a CoV S protein RBD (e.g., SARS-CoV-2 S protein RBD) in a biological sample according to a method described herein.

In one example, the antigen-binding protein of the disclosure is detectably labelled. Suitable detectable labels are known to the skilled person and described herein.

In one example, the kit comprises a positive control for CoV (e.g., SARS-CoV-2) and/or a negative control.

The present disclosure includes the following non-limiting Examples.

### EXAMPLES

### Example 1

### General Methods

### Monoclonal Antibody Production and Purification

DNA encoding antibody variable domains was amplified by PCR from the pHEN1 phage display vector and cloned into a human IgG1 expression vector based on pCEP4 (Invitrogen). After validation of the cloning by Sanger sequencing, the plasmids were transfected into ExpiCHO cells (Thermo Scientific) according to the manufacturer's protocol (1 μg DNA/ml of cells; 2:1 ratio of heavy chain to light chain) and following the max titer protocol. After 14 days, cell culture media were clarified by centrifugation and the IgG captured using Protein G resin (Genscript). IgG were eluted from the resin using 100 mM glycine pH 3.0, eluate was dialyzed against PBS the purity assessed by SDS-PAGE.

### Affinity Measurements Using Biolayer Interferometry (BLI)

Purified monoclonal antibodies (IgG) were buffer exchanged into PBS using equilibrated ZebaSpin columns (Thermo Fisher Scientific). The protein concentration was determined and the antibodies biotinylated by incubating for 30 min at room temperature with EZ-Link NHS-PEG4-Biotinylation reagent (Thermo Fisher Scientific) at a 10:1 biotin-to-protein ratio. Free biotin was removed from the samples by repeating the buffer exchange step in a second ZebaSpin column equilibrated with PBS. Affinity of interactions between biotinylated antibodies and purified soluble RBD proteins were measured Biolayer Interferometry (BLItz, ForteBio). Streptavidin biosensors were rehydrated in PBS containing 0.1% w/v BSA for 10 min at room temperature. Biotinylated antibody was loaded onto the sensors “on-line” using an advanced kinetics protocol, and global fits were obtained for the binding kinetics by running associations and dissociations of RBD proteins at a suitable range of molar concentrations (2-fold serial dilution ranging from 800 nM to 50 nM). The global dissociation constant (K_{D}) for each 1:1 binding interaction was determined using the BlitzPro 1.2.1.3 software.

### Polyclonal Phage and Monoclonal Soluble ELISA

For polyclonal ELISA, Maxisorp plates were coated with neutravidin overnight and 100 nM of biotinylated RBD was subsequently captured. 1×10⁹ purified phage were blocked in MPBST and incubated in each well for 1 h. Plates were washed with PBST, incubated with HRP-conjugated anti-M13 antibody (GE Healthcare) for 1 h and washed again. The plate was finally incubated with TMB substrate (Perkin Elmer), the reaction quenched with HCl and the plate read at Abs₄₅₀ₘₘ (ClarioStar—BMG Labtech). For monoclonal soluble ELISA, individual colonies from the selection titration plates were inoculated in 96 well plates and incubated at 37° C. overnight. The bacteria were re-inoculated the following day at 1:50 and incubated at 37° C. for 4 h. The plates were then spun down, the culture media discarded, bacteria resuspended in 2xYT supplemented with 100 μg/ml ampicillin and 1 mM IPTG and incubated overnight at 30° C. For ELISA, Maxisorp plates were coated with neutravidin overnight and 100 nM of biotinylated RBD subsequently captured. The plates were then incubated with 50 μl of culture media, clarified by centrifugation, for 1 h and then washed with PBST. The plates were subsequently incubated with HRP-conjugated chicken anti c-myc antibody (ICL Lab) for 1 h and washed again. The plate was finally incubated with TMB substrate (Perkin Elmer), the reaction quenched HCl and the plate read at Abs₄₅₀ₘₘ (ClarioStar—BMG Labtech).

Antigen production and purification

DNA encoding SARS-CoV-2 RBD (residues 319-541) was gene synthesized (Genscript) and cloned into pCEP4 mammalian expression vector with a N-terminal IgG leader sequence and C-terminal Avitag and His tag. The plasmid was transfected into Expi293 35 cells (Thermo Scientific) according to the manufacturer's protocol and the protein expressed for 7 days at 37° C., 5% CO₂. The cell culture was clarified by centrifugation, dialyzed with

PBS and the protein captured with Talon resin. The RBD was eluted with 150 mM imidazole in PBS, dialyzed with PBS and the purity assessed by visualization on SDS-PAGE. Homolog coronavirus RBD proteins (SARS-CoV-1, Bat, Pangolin) were expressed and purified as above.

### SARS -CoV-2 Neutralization Assays

Serial 2-fold dilutions of test monoclonal antibody are prepared in 96-well plates in octuplicate. The serial dilutions are incubated for 1 hour at 37° C. with an equal volume of SARS-CoV-2 isolate containing 200 TCID₅₀ (infectious dose). A Vero E6 suspension containing 2×10⁴ cells is added to each well, and plates are incubated at 37° C. (5% CO2). After 3 days, the plates are observed for cytopathic effect (CPE) and IC₅₀ values are calculated from four parameter dose-response curves (GraphPad Prism). All dilution steps of antibody, virus, and cells are performed in culture media containing MEM, 2% fetal bovine serum, and 1× penicillin-streptomycin-glutamine.

### Example 2

### Identification and Production of anti-SARS-CoV-2 Antibodies

This example describes experiments performed by the inventors to identify and produce antibodies binding a novel epitope of SARS-CoV-2 S protein RBD.

### Phage Display Selections

For phage display selection, SARS-CoV-2 RBD was biotinylated using a terminal AviTag and BirA biotin ligase (Avidity) according to the manufacturer's protocol and the Garvan-2 human antibody phage display library (Dudgeon et al., 2012, Rouet et al., 2017, Zeraati et al., 2018). Phage display selections were carried out by alternating between capture of the antigen on neutravidin coated wells on Maxisorp plates (Nunc) and streptavidin magnetic beads (Invitrogen) (Lee et al., 2007). For Maxisorp plate selection, neutravidin was coated overnight at 50 μg/mL in carbonate coating buffer, biotinylated RBD captured, and blocked in PBS supplemented with 0.1% Tween-20 and 4% skim milk (MPBST). 1×10¹² phage were blocked in MPBST, added to the wells containing antigen and incubated for 1 h. The wells were washed with 3×PBST, 1×PBS. Phage were eluted with 100 μg/mL trypsin for 1 h, then used to infect TG1 bacteria at an OD₆₀₀ₙₘ of 0.4. Infected TG1 were plated onto 2×YT agar plates supplemented with 100 μg/mL ampicillin and 2% glucose. For streptavidin beads selection, phages were blocked as described above and incubated with biotinylated RBD. 30 μl of streptavidin magnetic beads (Invitrogen) were blocked in PBST supplemented with 4% BSA (Sigma), then incubated for 15 min with the phage/antigen mix. Magnetic beads were washed with PBST and PBS and phage eluted as described above. 100 nM, 50 nM, 5 nM and 0.5 nM of biotinylated RBD was used for selection rounds 1 to 4. Phage titres used for selection were reduced to 1×10¹¹ for rounds 2 and 3 and 1×10¹⁰ for round 4. The number of PBST and PBS washes of bound phage was increased after each round.

### Results

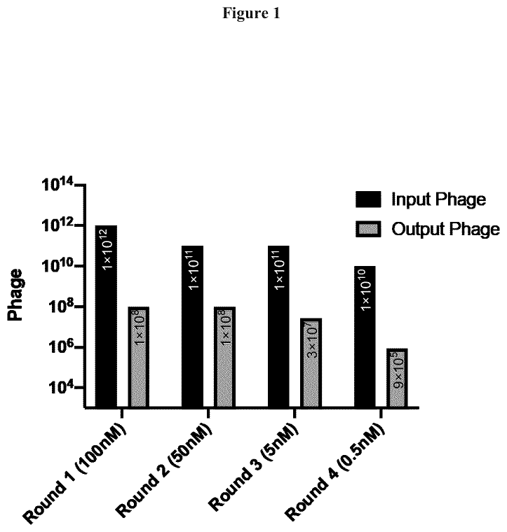

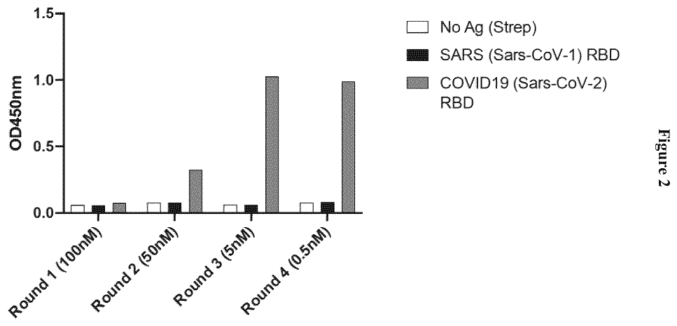

The initial phage display selections over the rounds of selection performed are illustrated in FIG. 1. Polyclonal phage ELISA (at OD450) with a naive selection, SARS-CoV-1 S protein RBD and SARS-CoV-2 S protein RBD showed increased enrichment for phage binding SARS-CoV-2 S protein RBD (FIG. 2).

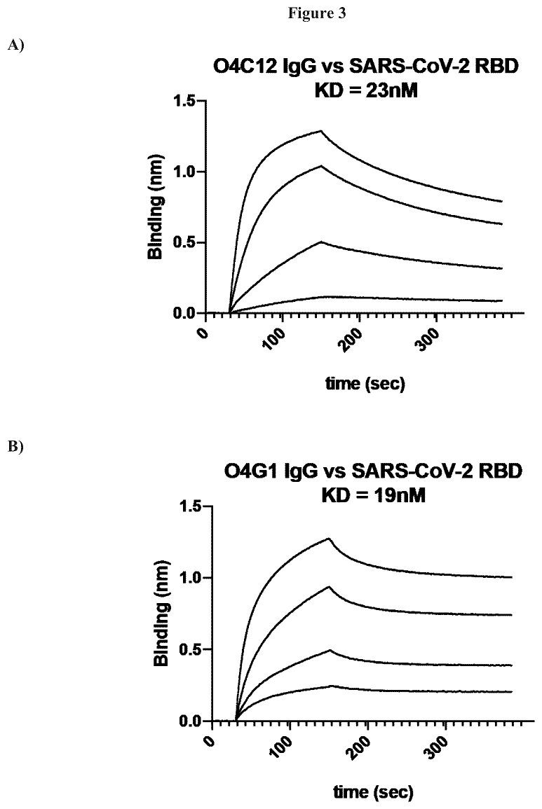

From the initial phage display selections, two parental clones designated O4C12 and O4G1 were selected based on their affinity for SARS-CoV-2 S protein RBD i.e., KD of 23 nM and 19 nM respectively (FIG. 3).

Neutralisation assays are performed for these antibodies against live SARS-CoV-2 to demonstrate that O4C12 and O4G1 neutralise SARS-CoV-2 infection of Vero E6 cells.

### Example 3

### Production of Affinity-Matured Variant Anti-SARS-CoV-2 Antibodies

This example describes experiments performed by the inventors to identify affinity matured variants of O4C12 and O4G1 having improved binding affinity for SARS-CoV-2 S protein RBD and cross-reactivity to SARS-CoV-1 S protein RBD.

### Generation and Selection of Affinity Matured Libraries

Antibody libraries for affinity maturation were generated by Kunkel mutagenesis according to previous protocols (Rouet et al., 2012) with the following modifications. Annealing of mutagenic oligonucleotide onto uracil containing single stranded DNA (dU-ssDNA) template was carried out in a molar ratio of 5:1 (oligo:template) with the addition of formamide (2% final). The reaction was heated to 90° C. for 2 mins before stepwise descent to 50° C. for 5 mins followed by further descent to 20° C. at a rate of 0.5° C./min using a thermocycler. Approximately 2 μg of purified covalently closed circular DNA (ccc-dsDNA) was concentrated to a final volume of 10 uL using speed vac centrifuge (V-AQ, 30° C. for 20 mins) for transformation with 100 uL of electrocompetent TG1 cells.

Selection for affinity matured variants was carried out using off-rate selections and streptavidin magnetic beads. Selections were performed essentially as previously described (Zahnd et al., 2010), with the following adjustments: phage were incubated with the biotinylated RBD for 1 h, excess unbiotinylated RBD was added (100× and 350× for rounds 2 and 3) and further incubated for 2/6 h for rounds 2/3 before capture on magnetic streptavidin beads. 50 nM, 25 nM, 10 nM of biotinylated RBD was used for selection rounds 1,2 and 3 respectively. Round 4 was a standard Maxisorp plate selection with 50 nM of monomeric full spike directly coated in carbonate coating buffer.

### Results

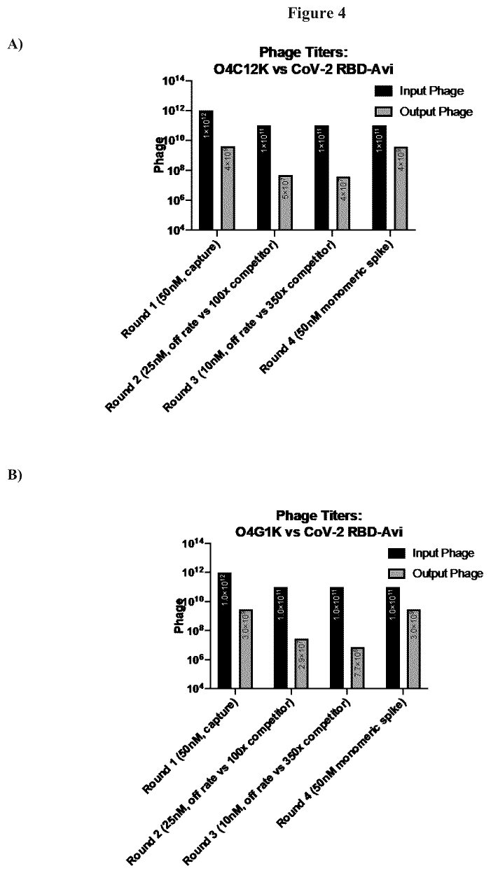

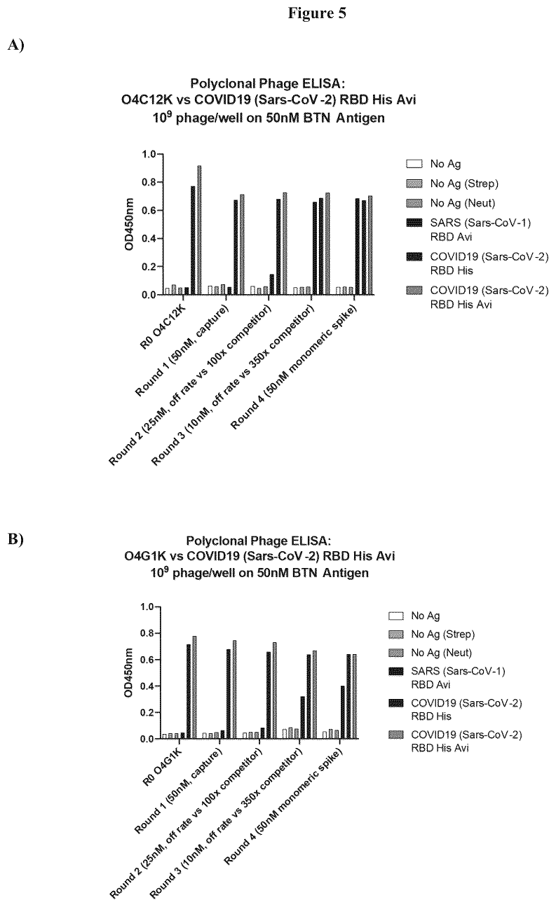

Results of the affinity maturation selections over four rounds of selection are illustrated in FIG. 4. Polyclonal phage ELISA (at OD450) with a naive selection, SARS-CoV-1 S protein RBD and SARS-CoV-2 S protein RBD showed increased enrichment for phage binding SARS-CoV-2 S protein RBD and cross-reactivity for SARS-CoV-1 S protein RBD (FIG. 5).

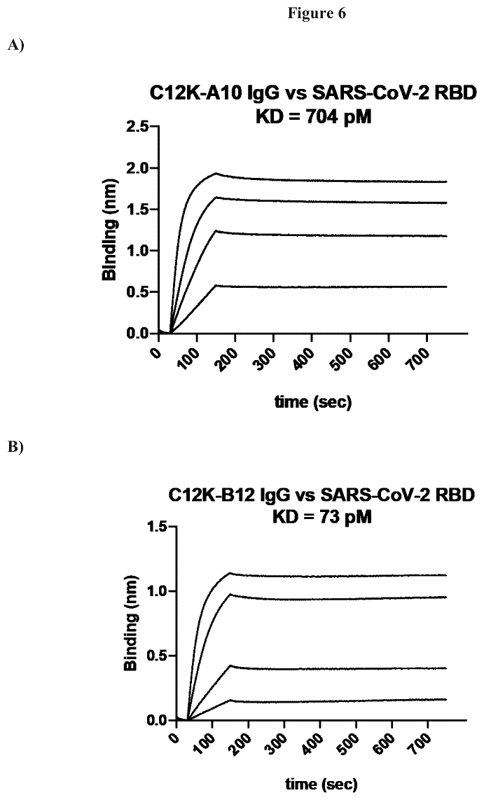

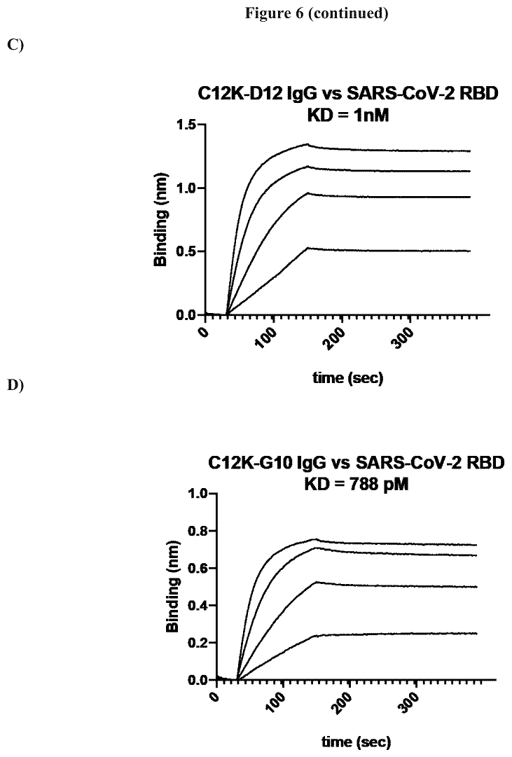

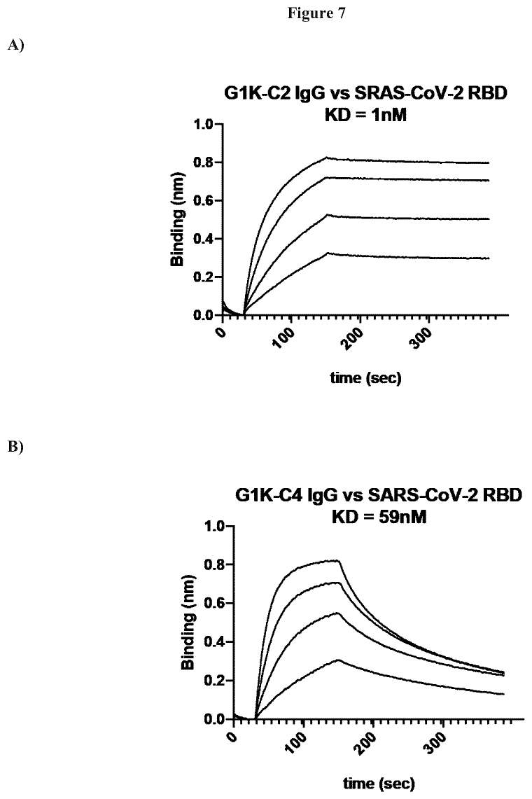

Based on affinity assays, four variants of O4C12 were selected (i.e., C12K-A10, C12K-B12, C12K-D12 and C12K-G10). These displayed a binding affinity (K_{D}) for SARS-CoV-2 S protein RBD of between 704 pM and 1nM (FIG. 6). Similarly. Two matured variants of O4G1 (i.e., G1K-C2 and G1K-C4) were selected based on binding affinity (K_{D}) for SARS-CoV-2 S protein RBD between 1 nM and 59 nM (FIG. 7).

Neutralisation assays are performed for the affinity matured antibodies against live SARS-CoV-2 to demonstrate that variants of O4C12 neutralise SARS-CoV-2 infection of Vero E6 cells.

### Example 4

### Epitope Mapping

DNA encoding SARS-CoV-2 S protein RBD (residues 319-541 of the S protein) carrying an 1150E mutation was cloned into pCEP4 mammalian expression vector with a N-terminal IgG leader sequence and C-terminal Avitag and His tag. The plasmid was transfected into Expi293 cells (Thermo Scientific) according to the manufacturer's protocol and the protein expressed for 7 days at 37° C., 5% CO₂. The cell culture was clarified by centrifugation, dialyzed with PBS and the protein captured with Talon resin. The RBD was eluted with 150 mM imidazole in PBS, dialyzed with PBS and the purity assessed by visualization on SDS-PAGE.

Purified monoclonal antibodies (IgG) were buffer exchanged into PBS using equilibrated ZebaSpin columns (Thermo Fisher Scientific). The protein concentration was determined and the antibodies biotinylated by incubating for 30 min at room temperature with EZ-Link NHS-PEG4-Biotinylation reagent (Thermo Fisher Scientific) at a 10:1 biotin-to-protein ratio. Free biotin was removed from the samples by repeating the buffer exchange step in a second ZebaSpin column equilibrated with PBS. Affinity of interactions between biotinylated antibodies and purified soluble RBD proteins were measured Biolayer Interferometry (BLItz, ForteBio). Streptavidin biosensors were rehydrated in PBS containing w/v BSA for 10 min at room temperature. Biotinylated antibody was loaded onto the sensors “on-line” using an advanced kinetics protocol, and global fits were obtained for the binding kinetics by running associations and dissociations of WT vs mutant RBD proteins at a suitable range of molar concentrations (2-fold serial dilution ranging from 800 nM to 50 nM). The mutant RBD proteins were as follows:
Mutant #445 comprising L137A/F138A double mutations*
Mutant #449 comprising T182A/N183A/Y187A triple mutations*
Mutant #459 comprising K6OS mutation*
Mutant #466 comprising I150E mutation*
   Mutation positioning is defined relative to the WT RBD sequence set forth in SEQ ID NO: 1.

The global dissociation constant (K_{D}) for each WT and I150E mutant 1:1 binding interaction was determined using the BlitzPro 1.2.1.3 software.

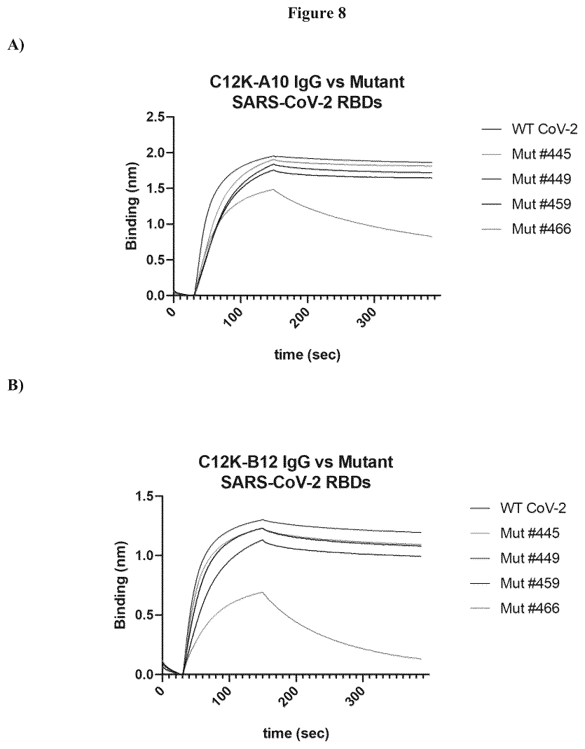

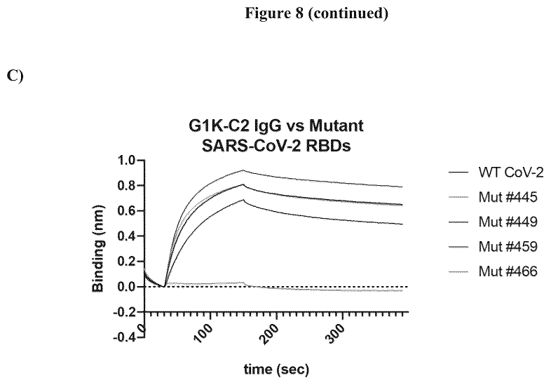

Results of the epitope mapping are illustrated in FIG. 8. These results show that when residue 150 of the SARS-CoV-2 S protein RBD was mutated from isoleucine (I) to Glutamic acid (E), binding of the anti-SARS-CoV antibodies was abolished. This data support the conclusion that the epitope to which these antibodies bind with the RBD of CoV S protein includes 150 and that the this residue is important for binding function.

### Example 5

### X-Ray Crystallography

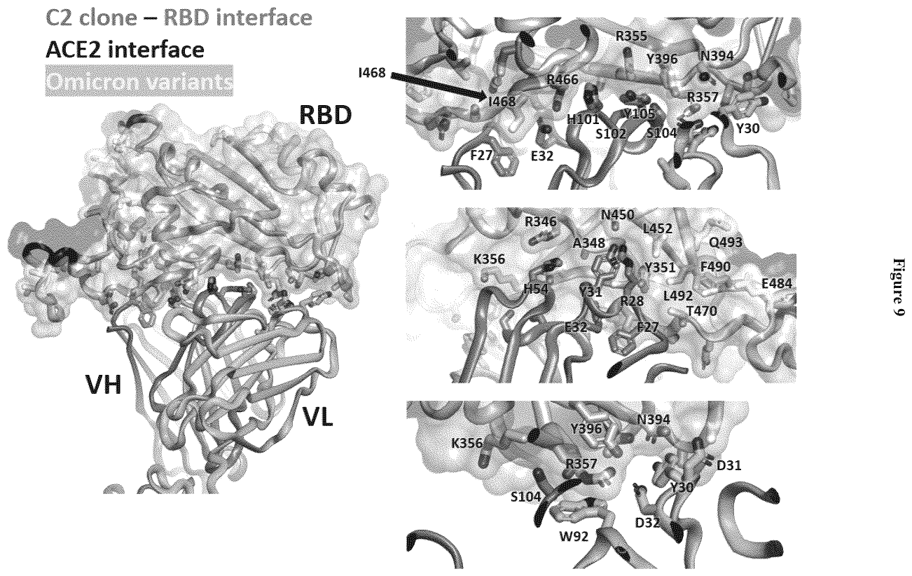

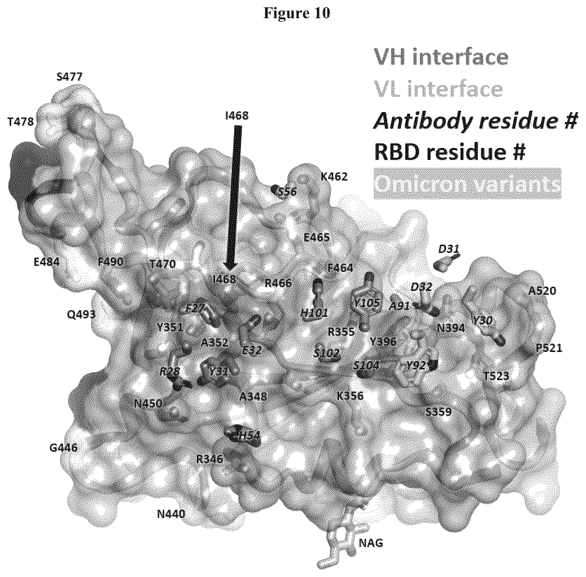

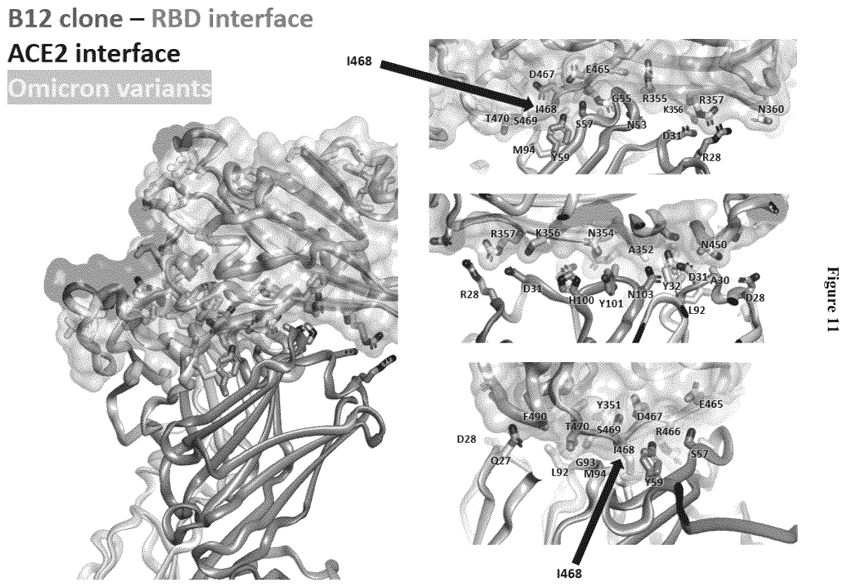

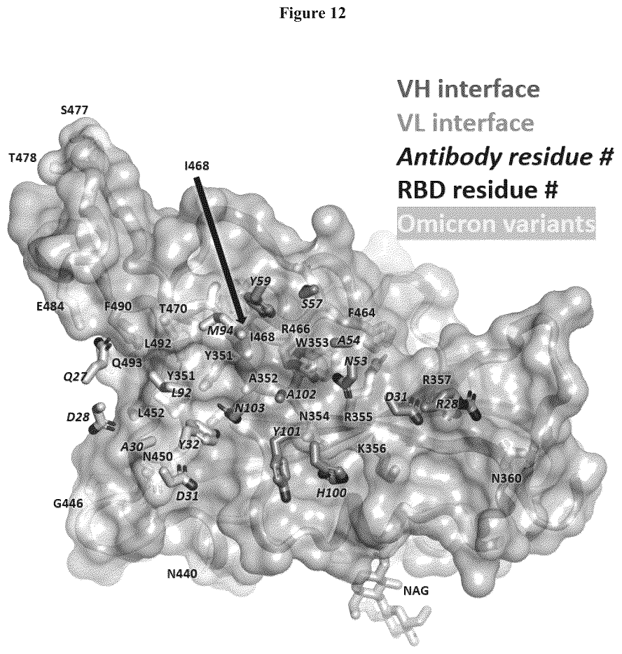

Crystallography was attempted with variants designated G1K-C2 and C12K-B12 in complex with the SARS-CoV-2 S protein RBD (i.e., residues 319-541 of the S protein) to characterize binding interaction of epitopes and paratopes. Crystals were formed for SARS-CoV-2 S protein RBD (SEQ ID NO: 1) in complex with a Fab containing G1K-C2 Fab heavy chain (SEQ ID NO: 15) and G1K-C2 light chain (SEQ ID NO: 16). Crystals were also formed for SARS-CoV-2 S protein RBD (SEQ ID NO: 1) in complex with a Fab containing C12K-B12 Fab heavy chain (SEQ ID NO: 9) and C12K-B12 light chain (SEQ ID NO: 10). Structures were solved for these two complexes using X-ray crystallography. These structures are illustrated in FIGS. 9-12, with residues of the Fabs shown to be in close proximity to the surface of the S protein RBD indicated.

These crystal structures support the conclusion that the epitope to which G1K-C2 and C12K-B12 bind with the RBD of CoV S protein includes residue 150 (residue 468 of the full length S protein as shown in FIGS. 9-12) and that the this residue is important for binding function.

Selected references from Examples 1-3

Dudgeon et al., (2012) Methods Mol Biol, 911:383-397.

Lee et al., (2007) Nat Protoc, 2:3001-3008.

Rouet et al., (2012) Methods Mol Biol, 907:195-209

Rouet et al., (2017) PNAS, USA, 114:3897-3902.

Zahnd et al., (2010) Protein Eng Des Sel, 23:175-184

Zeraati et al., (2018) Methods Mol Biol, 1827:197-209

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An antigen-binding protein comprising an antibody variable region which binds to a coronavirus (CoV) spike (S) protein receptor binding domain (RBD), wherein the antibody variable region binds to an epitope of the CoV S protein RBD comprising at least residue 150 numbered relative to the severe acute respiratory syndrome (SARS) CoV-2 S protein RBD amino acid sequence set forth in SEQ ID NO: 1.

2. The antigen-binding protein of claim 1, wherein the antibody variable region binds at least residue 1501 within the epitope of the CoV S protein RBD, wherein residue 1501 is numbered relative to the SARS-CoV-2 S protein RBD amino acid sequence set forth in SEQ ID NO: 1.

3. The antigen-binding protein of claim 1 or 2, wherein said antigen-binding protein binds SARS-CoV-2 S protein RBD with an equilibrium binding constant (K_{D}) of about 60 nM or better.

4. The antigen-binding protein of any one of claims 1 to 3, wherein said antigen-binding protein neutralises coronavirus infection of mammalian cells.

5. The antigen-binding protein of claim 4, wherein said antigen-binding protein neutralises SARS-CoV-2 infection of mammalian cells.

6. The antigen-binding protein of any one of claims 1 to 5, wherein said antigen-binding protein neutralises SARS-CoV-2 infection of Vero E6 cells with a half-maximal inhibitory concentration (IC50) of about 50 μg/mL or better.

7. The antigen-binding protein of any one of claims 1 to 6, wherein said antigen-binding protein comprises an antibody variable region which binds to the same epitope within a coronavirus S protein RBD as that bound by an antibody selected from:
(i) an antibody comprising a heavy chain variable domain (V_{H}) comprising the sequence set forth in SEQ ID NO: 3 and a light chain variable domain (V_{L}) comprising the sequence set forth in SEQ ID NO: 4;
(ii) an antibody comprising a V_{H} comprising the sequence set forth in SEQ ID NO: 7 and a V_{L} comprising the sequence set forth in SEQ ID NO: 8;
(iii) an antibody comprising a V_{H} comprising the sequence set forth in SEQ ID NO: 9 and a V_{L} comprising the sequence set forth in SEQ ID NO: 10;
(iv) an antibody comprising a V_{H} comprising the sequence set forth in SEQ ID NO: 11 and a V_{L} comprising the sequence set forth in SEQ ID NO: 12;
(v) an antibody comprising a V_{H} comprising the sequence set forth in SEQ ID NO: 13 and a V_{L} comprising the sequence set forth in SEQ ID NO: 14;
(vi) an antibody comprising a V_{H} comprising the sequence set forth in SEQ ID NO: 5 and a V_{L} comprising the sequence set forth in SEQ ID NO: 6;
(vii) an antibody comprising a V_{H} comprising the sequence set forth in SEQ ID NO: 15 and a V_{L} comprising the sequence set forth in SEQ ID NO: 16; and
(viii) an antibody comprising a V_{H} comprising the sequence set forth in SEQ ID NO: 17 and a V_{L} comprising the sequence set forth in SEQ ID NO: 18.

8. The antigen-binding protein of any one of claims 1 to 7, wherein said antigen-binding protein comprises an antibody variable region comprising:
(i) a V_{H} comprising complementarily determining region (CDR) 1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 22, 23 and 24 respectively;
(ii) a V_{H} comprising CDR1, CDR2 and CDR3 comprising the sequences set forth in SEQ ID NOs: 19, 20 and 21 respectively, and a V_{L} comprising CDR1, CDR2 and CDR3 comprising the sequences set forth in SEQ ID NOs: 31, 23 and 32 respectively;
(iii) a V_{H} comprising CDR1, CDR2 and CDR3 comprising the sequences set forth in SEQ ID NOs: 19, 20 and 33 respectively, and a V_{L} comprising CDR1, CDR2 and CDR3 comprising the sequences set forth in SEQ ID NOs: 34, 23 and 24 respectively;
(iv) a V_{H} comprising CDR1, CDR2 and CDR3 comprising the sequences set forth in SEQ ID NOs: 19, 20 and 35 respectively, and a V_{L} comprising CDR1, CDR2 and CDR3 comprising the sequences set forth in SEQ ID NOs: 22, 23 and 24 respectively;
(v) a V_{H} comprising CDR1, CDR2 and CDR3 comprising the sequences set forth in SEQ ID NOs: 19, 20 and 36 respectively, and a V_{L} comprising CDR1, CDR2 and CDR3 comprising the sequences set forth in SEQ ID NOs: 37, 38 and 24 respectively;
(vi) a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 30 respectively;
(vii) a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 28, 29 and 39 respectively; or
(viii) a V_{H} comprising CDR1, CDR2 and CDR3 comprising the sequences set forth in SEQ ID NOs: 25, 26 and 27 respectively, and a V_{L} comprising CDR1, CDR2 and CDR3 comprising the sequences set forth in SEQ ID NOs: 40, 29 and 30 respectively.

9. The antigen-binding protein of any one of claims 1 to 8, wherein said antigen-binding protein comprises an antibody variable region comprising:
(i) a V_{H} comprising a sequence which is at least 90% identical to the sequence set forth in SEQ ID NO: 3 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical to the sequence set forth in SEQ ID NO: 4 provided that the V_{L} comprises a CDR1 set forth in SEQ ID NO: 22, a CDR2 set forth in SEQ ID NO: 23 and a CDR3 set forth in SEQ ID NO: 24;
(ii) a V_{H} comprising a sequence which is at least 90% identical to the sequence set forth in SEQ ID NO: 7 provided that the V_{H} comprises a CDR1 set forth in SEQ ID NO: 19, a CDR2 set forth in SEQ ID NO: 20 and a CDR3 set forth in SEQ ID NO: 21, and a V_{L} comprising a sequence which is at least 90% identical to the sequence set forth in SEQ ID NO: 8 provided that the V_{L} comprises a CDR1 set forth in SEQ ID NO: 31, a CDR2 set forth in SEQ ID NO: 23 and a CDR3 set forth in SEQ ID NO: 32;
(iii) a V_{H} comprising a sequence which is at least 90% identical to the sequence set forth in SEQ ID NO: 9 provided that the V_{H} comprises a CDR1 set forth in SEQ ID NO: 19, a CDR2 set forth in SEQ ID NO: 20 and a CDR3 set forth in SEQ ID NO: 33, and a V_{L} comprising a sequence which is at least 90% identical to the sequence set forth in SEQ ID NO: 10 provided that the V_{L} comprises a CDR1 set forth in SEQ ID NO: 33, a CDR2 set forth in SEQ ID NO: 23 and a CDR3 set forth in SEQ ID NO: 24;
(iv) a V_{H} comprising a sequence which is at least 90% identical to the sequence set forth in SEQ ID NO: 11 provided that the V_{H} comprises a CDR1 set forth in SEQ ID NO: 19, a CDR2 set forth in SEQ ID NO: 20 and a CDR3 set forth in SEQ ID NO: 35, and a V_{L} comprising a sequence which is at least 90% identical to the sequence set forth in SEQ ID NO: 12 provided that the V_{L} comprises a CDR1 set forth in SEQ ID NO: 22, a CDR2 set forth in SEQ ID NO: 23 and a CDR3 set forth in SEQ ID NO: 24;
(v) a V_{H} comprising a sequence which is at least 90% identical to the sequence set forth in SEQ ID NO: 13 provided that the V_{H} comprises a CDR1 set forth in SEQ ID NO: 19, a CDR2 set forth in SEQ ID NO: 20 and a CDR3 set forth in SEQ ID NO: 36, and a V_{L} comprising a sequence which is at least 90% identical to the sequence set forth in SEQ ID NO: 14 provided that the V_{L} comprises a CDR1 set forth in SEQ ID NO: 37, a CDR2 set forth in SEQ ID NO: 38 and a CDR3 set forth in SEQ ID NO: 24;
(vi) a V_{H} comprising a sequence which is at least 90% identical to the sequence set forth in SEQ ID NO: 5 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical to the sequence set forth in SEQ ID NO: 6 provided that the V_{L} comprises a CDR1 set forth in SEQ ID NO: 28, a CDR2 set forth in SEQ ID NO: 29 and a CDR3 set forth in SEQ ID NO: 30;
(vii) a V_{H} comprising a sequence which is at least 90% identical to the sequence set forth in SEQ ID NO: 15 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical to the sequence set forth in SEQ ID NO: 16 provided that the V_{L} comprises a CDR1 set forth in SEQ ID NO: 28, a CDR2 set forth in SEQ ID NO: 29 and a CDR3 set forth in SEQ ID NO: 39; or
(viii) a V_{H} comprising a sequence which is at least 90% identical to the sequence set forth in SEQ ID NO: 17 provided that the V_{H} comprises a CDR1 set forth in SEQ ID NO: 25, a CDR2 set forth in SEQ ID NO: 26 and a CDR3 set forth in SEQ ID NO: 27, and a V_{L} comprising a sequence which is at least 90% identical to the sequence set forth in SEQ ID NO: 18 provided that the V_{L} comprises a CDR1 set forth in SEQ ID NO: 40, a CDR2 set forth in SEQ ID NO: 29 and a CDR3 set forth in SEQ ID NO: 30.

10. The antigen-binding protein of any one of claims 1 to 9, wherein said antigen-binding protein comprises an antibody variable region comprising:
(i) a V_{H} comprising the sequence set forth in SEQ ID NO: 3 and a V_{L} comprising the sequence set forth in SEQ ID NO: 4;
(ii) a V_{H} comprising the sequence set forth in SEQ ID NO: 7 and a V_{L} comprising the sequence set forth in SEQ ID NO: 8;
(iii) a V_{H} comprising the sequence set forth in SEQ ID NO: 9 and a V_{L} comprising the sequence set forth in SEQ ID NO: 10;
(iv) a V_{H} comprising the sequence set forth in SEQ ID NO: 11 and a V_{L} comprising the sequence set forth in SEQ ID NO: 12;
(v) a V_{H} comprising the sequence set forth in SEQ ID NO: 13and a V_{L} comprising the sequence set forth in SEQ ID NO: 14;
(vi) a V_{H} comprising the sequence set forth in SEQ ID NO: 5 and a V_{L} comprising the sequence set forth in SEQ ID NO: 6;
(vii) a V_{H} comprising the sequence set forth in SEQ ID NO: 15 and a V_{L} comprising the sequence set forth in SEQ ID NO: 16; or
(viii) a V_{H} comprising the sequence set forth in SEQ ID NO: 17 and a V_{L} comprising the sequence set forth in SEQ ID NO: 18.

11. The antigen-binding protein of any one of claims 1 to 10, wherein the V_{H} and the V_{L} are in a single polypeptide chain.

12. The antigen-binding protein of claim 11, wherein the antigen-binding protein is:
(i) a single chain FAT fragment (scFv);
(ii) a dimeric scFv (di-scFv); or
(iii) at least one of (i) and/or (ii) linked to a Fc or a heavy chain constant domain (CH) 2 and/or C_{H}3.

13. The antigen-binding protein of claim 11 or claim 12, wherein the V_{H} and the V_{L} are in separate polypeptide chains.

14. The antigen-binding protein of claim 13, wherein the antigen-binding protein is:
(i) a diabody;
(ii) a triabody;
(iii) a tetrabody;
(iv) a Fab;
(v) a F(ab′)₂;
(vi) a Fv; or
(iv) one of (i) to (iii) linked to a Fc or a heavy chain constant domain (CH) 2 and/or C_{H}3.

15. The antigen-binding protein of claim 14, which is an antibody.

16. The antigen-binding protein of claim 15, wherein the antigen-binding protein is chimeric, CDR grafted, de-immunized, humanized, synhumanized, primatized or human.

17. The antigen-binding protein of claim 15, wherein the antigen-binding protein is a human antibody.

18. The antigen-binding protein of any one of claims 1 to 17, wherein the antigen-binding protein is conjugated to a compound.

19. The antigen-binding protein of claim 18, wherein the compound is selected from the group consisting of a detectable label, a therapeutic compound, a nucleic acid, a peptide, a protein, a compound that increases the half-life of the protein in a subject and mixtures thereof.

20. The antigen-binding protein of claim 19, wherein the detectable label is selected from the group consisting of: a radiolabel, a fluorescent label, an enzymatic label and an imaging agent.

21. One or more nucleic acids encoding an antigen-binding protein of any one of claims 1 to 17.

22. An expression construct comprising the one or more nucleic acid of claim 21.

23. A host cell comprising the one or more nucleic acids of claim 21, wherein said cell is capable of expressing an antigen-binding protein of any one of claims 1 to 17.

24. A composition comprising the antigen-binding protein of any one of claims 1 to 19 and an acceptable carrier.

25. A method of treating or preventing infection with a coronavirus (CoV) and/or acute respiratory syndrome caused by infection with a CoV in a subject in need thereof, the method comprising administering to the subject the antigen-binding protein of any one of claims 1 to 20 or the composition of claim 24.

26. The method of claim 25, wherein administration of the antigen-binding protein to the subject neutralises the coronavirus thereby preventing or reducing infection in the subject.

27. A method of detecting the presence or absence of a coronavirus (CoV) spike (S) protein receptor binding domain (RBD) in a sample, said method comprising:
(i) contacting the sample with an antigen-binding protein of any one of claims 1 to 20; and
(ii) analysing the sample for binding between CoV S protein RBD and the antigen-binding protein.

28. A method of diagnosing coronavirus (CoV) infection in a subject, the method comprising:
(i) performing a method of claim 27 on a sample obtained from the subject to determine the presence or absence of CoV S protein RBD in the sample; and
(ii) diagnosing whether or not the subject is infected with coronavirus based on the presence or absence of the CoV S protein RBD in the sample.

29. The method of claim 27 or 28, wherein the method is performed in vitro and the sample is, or is obtained from, a nasopharyngeal swab, a oropharyngeal swab, a nasal aspirate, a nasal wash, saliva, sputum, tracheal aspirate or bronchoalveolar lavage (BAL).

30. The method of any one of claims 25 to 29, wherein the CoV is a severe acute respiratory syndrome (SARS) CoV-2.

31. The method of any one of claims 25 to 29, wherein the CoV is a severe acute respiratory syndrome (SARS) CoV-1.

32. Use of an antigen-binding protein of any one of claims 1 to 20 or a composition of claim 24 to treat or prevent infection with a coronavirus (CoV) and/or acute respiratory syndrome caused by infection with a CoV in a subject.

33. Use of an antigen-binding protein of any one of claims 1 to 20 to detect coronavirus (CoV) spike (S) protein, optionally wherein the antigen-binding protein is detectably labelled.

34. Use of an antigen-binding protein of any one of claims 1 to 20 or a nucleic acid of claim 21 or an expression construct of claim 22 or a host cell of claim 23 or a composition of claim 24 in the preparation of a medicament for treating and/or preventing infection with a coronavirus (CoV) and/or acute respiratory syndrome caused by infection with a CoV in a subject in need thereof The use of claim 33 or 34, wherein the CoV is a severe acute respiratory syndrome (SARS) CoV-2.

36. The use of claim 33 or 34, wherein the CoV is a severe acute respiratory syndrome (SARS) CoV-1.

37. The antigen-binding protein of any one of claims 1 to 20 or the composition of claim 24 for use in treating and/or preventing infection with a coronavirus (CoV) and/or acute respiratory syndrome caused by infection with a CoV in a subject in need thereof.

38. The antigen-binding protein for use according to claim 37, wherein the CoV is a severe acute respiratory syndrome (SARS) CoV-2.

39. The antigen-binding protein for use according to claim 37, wherein the CoV is a severe acute respiratory syndrome (SARS) CoV-1.
